SEQUENCE LISTING (4 sequences)

SEQ ID NO: 1 - DNA, 29 bp, Artificial
  synthetic JUN1 degenerate primer sequence
cccaagcttgcatgcgaattcnnnncagg

SEQ ID NO: 2 - DNA, 21 bp, Artificial
  synthetic JUN15 primer
cccaagcttgcatgcgaattc

SEQ ID NO: 3 - DNA, 19 bp, Homo sapiens
ggggatcgttatggaaaga

SEQ ID NO: 4 - DNA, 21 bp, Homo sapiens
tccattccactcgtttccttt

(19) United States
(12) Patent Application Publication
(10) Pub. No.: US 2009/0098534 A1
(43) Pub. Date: Apr. 16, 2009

(54) Full Karyotype Single Cell Chromosome Analysis

(75) Inventors: Heinz-Ulrich G. Weier, El Cerrito, CA (US); Santiago Munne, Short Hills, NJ (US)

Correspondence Address: LAWRENCE BERKELEY NATIONAL LABORATORY, ONE CYCLOTRON ROAD, MAIL STOP 90B, UNIVERSITY OF CALIFORNIA, BERKELEY, CA, 94720, US

(73) Assignee: The Regents of the University of California, Oakland, CA (US)

(21) Appl. No.: 11/817,072

(22) PCT Filed: Feb. 27, 2006
(86) PCT No.: PCT/US06/07335

§ 371 Date: Aug. 24, 2007

Related U.S. Application Data
(60) Provisional application No. 60/656,615, filed on Feb. 25, 2005.
(60) Provisional application No. 60/658,778, filed on Mar. 4, 2005.

(51) Int. Cl. C12Q 1/68; C07H 21/04

(52) U.S. Cl. 435/6; 536/24.31

(57) ABSTRACT

A full set of 24 chromosome-specific probes to analyze single cells or cell organelles to test for abnormalities is described. When used in an assay based on sequential hybridization, the full set is comprised of three subsets of chromosome-specific probes with each set comprised of 8 different probes. Also described are assays using a set of probes to analyze single cells and cellular organelles to accurately determine the number and type of targeted human chromosomes in various types of cells and cell organelles, such as tumor cells, interphase cells and first polar bodies biopsied from non-inseminated oocytes. Methods of selection or generation of suitable probes and hybridization protocols are described, as are preferred probes for frill set of 24 chromosome-specific probes to target all 24 human chromosomes are described in the Tables.

### CROSS-REFERENCE TO RELATED APPLICATIONS

This application is the U.S. national phase application of International Application No. PCT/US2006/007335, filed on Feb. 27, 2006, also claiming priority to U.S. Provisional Patent Application No. 60/656,615, filed on Feb. 25, 2005, and U.S. Provisional Patent Application No. 60/658,778, filed on Mar. 4, 2005, all of which are hereby incorporated by reference in their entirety.

### STATEMENT OF GOVERNMENTAL SUPPORT

This invention was made during work supported by U.S. Department of Energy under Contract No. DE-AC03-76SF00098, now Contract No. DE-AC02-05CH11231. The government has certain rights in this invention.

### REFERENCE TO SEQUENCE LISTING AND TABLE APPENDIX

Applicants incorporate by reference the attached sequence listing found in paper form, which is identical to the copy found in computer readable form. Applicants also incorporate by reference the attached Table 3 found in the Table Appendix.

### BACKGROUND OF THE INVENTION

1. Field of the Invention

The present invention relates to chromosomal analysis for abnormalities of human oocytes, polar bodies or blastomeres using fluorescence hybridization and spectral imaging analysis.

2. Related Art

In vitro fertilization (IVF) has been able to help thousands of American couples to conceive. However, the national average pregnancy rate after IVF is only 24.9% (most recent available data from 1998) and it drops with maternal age, from 40% at age 30 to 21.6% in women 40 years of age and to 6% in 43 years old women. High pregnancy rates are very important, since each IVF cycle is very costly and presents a highly stressful situation to the couple. Major causes of a low pregnancy rate in patients of advanced maternal age are numerical chromosome aberrations, which result in failed fertilizations, embryos not implanting or embryo loss after implantation.

In general, chromosome abnormalities are the major cause of reproductive failure, with an incidence of 21% in spontaneous abortions (Hassold et al. 1980, Warburton et al. 1980, 1986). Of these, numerical aberrations involving gonosomes and chromosomes 21, 18, 16 and 13 account for 50% of chromosomally abnormal abortions. In contrast to single gene defects, numerical chromosome abnormalities frequently occur de novo. The only risk factor known is maternal age, with the incidence of trisomy detected by amniocentesis increasing from 0.6% to 2.2% from age 35 to age 40 (Hook et al. 1992). Thus, the screening of chromosome aneuploidies in human embryos by fluorescence in situ hybridization (FISH) using X, Y, 18, 13 and 21 probes should significantly reduce the risk of older IVF patients delivering trisomic offspring. Ploidy assessment of single blastomeres by FISH was first achieved in a time frame compatible with IVF in the early 1990's (Grifo et al. 1990, 1992; Munné et al. 1993). In those studies it was hypothesized that preimplantation genetic diagnosis (PGD) of numerical chromosome abnormalities may increase the pregnancy rate in women of advanced maternal age who are undergoing IVF.

The causes of the decline in embryo implantation observed with increasing maternal age are still under debate. Some investigators propose that maternal age affects uterine receptivity, while others suggest that it mostly affects oocyte viability. Nevertheless, the high implantation rates obtained using donated oocytes, where some control can be exerted over donor age and uterine factors could be measured, strongly indicate that the oocyte is the major cause of age-related implantation failure (Navot et al. 1994). Ooplasmic components may be involved (Keefe et al. 1995, Cohen et al. 1998, Barrit et al. 2000), as well as zona pellucida thickening (Cohen 1993; Meldrum et al. 1998), but the clearest link so far between maternal age and embryo competence is aneuploidy. The increase in aneuploidy with maternal age in spontaneous abortuses and live offspring was also found in both cleavage-stage embryos (Munné et al. 1995, Marquez et al. 2000) and unfertilized oocytes (Dailey et al. 1996). The rate of chromosomal abnormalities in IVF embryos was higher than that reported in spontaneous abortions, suggesting that a sizable part of chromosomally abnormal embryos are eliminated before clinical recognition of pregnancy. Such loss of embryos could account for the decline in implantation with maternal age. For instance, the rates of embryonic monosomy and trisomy are similar (Munné et al. 1995), while with the exception of monosomy 21 ( 1/1,000 karyotyped abortions), the other autosomal monosomies are normally undetected in clinically recognized pregnancies. Furthermore, monosomies in mice (Magnuson et al. 1985) and human (Sandalinas et al. 2001) do not develop to blastocyst stage, with the exception of human monosomy 21 and X. This is in agreement with the observation that blastocyst formation declines with maternal age in women over 30 (Janny and Menezo 1996). It is not known whether trisomies that develop to term (13, 18, 21) have a lower implantation rate than normal embryos. However, even recognized pregnancies with trisomy 21 spontaneously abort in 84% to 93% of cases depending on the age of the woman (Warburton et al. 1986).

Recognizing the correlation between aneuploidy and declining implantation rates with maternal age, it was surmised that in patients undergoing in vitro fertilization (IVF) a negative selection against chromosomally abnormal embryos could reverse this trend (Munné et al. 1993). Currently, negative selection of aneuploid embryos can only be done through PGD, either by polar body or blastomere analysis. Low metaphase yield and less than 30% of karyotypable metaphases together with the requirement of overnight culture in antimitotics (Santaló et al. 1995) make karyotype analysis unsuitable for PGD. FISH allows chromosome enumeration to be performed on interphase cell nuclei, i.e. without the need for culturing cells or preparing metaphase spreads. FISH has been applied to PGD of common aneuploidies using either human blastomeres (cells from 2- to 16-cell stage embryos) or oocyte polar bodies (Munné et al. 1993, 1995a, 1995b, 1998a, 1998b, 1998c, Munné and Weier 1996, Verlinsky et al. 1995, Verlinsky and Kuliev 1996, Verlinsky et al. 1998a,b, Gianiaroli et al. 1997, 1999). Currently, probes for chromosomes X, Y, 13, 14, 15, 16, 18, 21 and 22 are being used in 2 rounds of hybridizations (Bahçe et al. 2000), with the potential of detecting 70% of the aneuploidies involved in spontaneous abortions.

PGD of aneuploidy is based on the analysis of polar bodies biopsied from unfertilized oocytes or zygotes or the analysis of a single blastomere removed from a 6- to 8-cell stage human embryo. Only those eggs (or embryos) considered chromosomally normal are replaced to women undergoing in vitro fertilization in order to reduce embryo wastage and prevent trisomic offspring. Because only one single cell can be analyzed and this is in interphase and cannot be cultured to obtain a metaphase cell, PGD of aneuploidy is currently performed by FISH. However, current FISH technology can only detect a very limited number of chromosome abnormalities in interphase cells because of a lack of a large number of sufficiently different fluorochromes.

Current commercially available FISH multiprobe combinations consist of only 5 chromosome types (X, Y, 13, 18, 21 or 13, 16, 18, 21, 22). Previous studies (Munné et al. 1999, Gianaroli et al. 1999, Jobanputra et al. 2002) indicated that at least 8 probes are necessary to significantly reduce embryo wastage. Existing technology thus required two rounds of hybridization to score 8-10 chromosomes, which is time consuming. It would be advantageous to perform a comprehensive cytogenetic analysis of human oocytes by counting all chromosome contained in the first polar body (1 Pb) using new technology.

Mammalian eggs are composed of the MII-oocyte and the first polar body (1 Pb). The 1 Pb is not essential for embryo development. The first polar body and the MII oocytes are the products of the first mitotic division. Thus, if there was non-disjunction or abnormal segregation of chromatids and an extra chromosome or chromatid was found in the first polar body, one should be missing in the MII oocyte, and vice versa. Knowing the chromosomes contained in the 1 Pb allows one to judge the chromosomal make-up of the corresponding oocytes.

First polar bodies (1 Pbs) are not involved in embryo development and can be removed from eggs prior to fertilization. The analysis of 1 Pbs allows one to judge the chromosomal composition of the egg, since the number of chromosomes in 1 Pbs and oocytes usually adds up to a normal diploid complement. This strategy of pre-fertilization genetic analysis is now pursued in several major IVF Centers in the US. However, present approaches to genetic analysis rely on careful timing of 1 Pb harvests since polar bodies contain condensed chromatin only for a short time after biopsy (Marquez et al., 1998). Another rather laborious approach uses fusion of single blastomeres with mouse zygotes or cattle oocytes to induce chromatin condensation so that chromosomes can be identified by banding or FISH painting using whole chromosome painting probes (Verlinsky and Evsikov, 1999, Willadsen et al., 1999).

With most cells available for analysis likely to be in interphase, we recognized a need to develop a rapid and innovative chromosome enumeration protocol based on the hybridization of a larger set of chromosome-specific probes. It seems the recently developed spectral imaging (SIm) approach will provide the technological basis to achieve this goal.

The recent introduction of spectroscopic detection methods in fluorescence in situ hybridization (FISH) assays now allows the simultaneous use of many more uniquely labeled DNA probes than before. This technique, in which single band pass emission filters are replaced by a multi band pass filter and an interferometer, has been termed ‘Spectral Imaging’ (Schlöck et al., 1996). Spectral imaging technology can now be installed in any laboratory equipped with a fluorescence microscope.

### BRIEF SUMMARY OF THE INVENTION

The present invention provides a full set of 24 chromosome-specific probes to analyze single cells to test for abnormalities in all 24 human chromosomes. In a preferred embodiment, a full set of 24 chromosome-specific probes described in Table 2. In another preferred embodiment, a full set of 24 chromosome-specific probes as described in Table 3.

The present invention further provides a first subset of 8 chromosome-specific probes to analyze single cells. In one aspect, single cells and cellular organelles which can be used for the present hybridization analysis include but are not limited to, blastomeres, spermatocytes, somatic cells, interphase cells, tumor cells, first polar bodies (1 Pbs) and second polar bodies.

In another aspect, the invention provides for a method of fluorescence in situ hybridization (FISH) to accurately determine the number and type of all human chromosomes in interphase cells and first polar bodies biopsied from non-inseminated oocytes using the full set of three subsets of 8-chromosome-specific probes described in Table 2 or 3. The assay is based on sequential hybridization of three sets of chromosome-specific DNA probes with each set comprised of 8 different probes. Also described are methods of selection or generation of suitable probe DNAs, non-isotopical labeling of DNA with fluorescent haptens and optimization of hybridization protocols.

It is an object of the invention to provide the full set of 24 chromosome-specific probes for analysis to determine chromosomal abnormality prior to fertilization or implantation during the course of in vitro fertilization or to determine the chromosome-specific rates of aneuploid cells for various age groups.

The present invention also describes methods for chromosomal analysis of tumor cells. The full set of probes described in Table 2 or 3 can be used to analyze tumor cells for chromosomal abnormality or at least 24 probes directed to several different target loci or amplicons may be developed and used as three 8-probe subsets using the nine-color, eight target scheme in Table 1, to analyze a tumor cell to provide a chromosomal profile of the cancer.

The present invention also provides a method for a cytogenetic test based on hybridization of DNA probes and detection by spectral imaging to detect numerical chromosome aberration involving any of the 24 different human chromosome types comprising the steps of: (a) providing a single cell or organelle; (b) treating the single cell and fixing it to a substrate for analysis to increase accessibility of target DNA and to reduce nonspecific binding; (c) providing a first set of 8 probes to detect the target chromosomes; (d) hybridization of the probes to the target chromosomes in the single cell or organelle; (e) posthybridization washes to remove unbound probes and post hybridization processing such as washes, blocking, detection and amplification; and (f) detecting the hybridized probes to the target chromosomes carried out such that numerical chromosome aberration involving any of the 24 different human chromosome types can be detected. The steps are repeated for second and third subsets each comprising 8 chromosome-specific probes. In a preferred embodiment, the detection step (f) is performed using a filter-based fluorescent microscope, optionally equipped with a spectral imaging system.

In another embodiment, the cytogenetic test is performed on interphase cells. In another embodiment, the cytogenetic test is performed on interphase, non-proliferating or resting cells or organelles.

### BRIEF DESCRIPTION OF THE DRAWINGS

FIG. 1 is a schematic of the Applied Spectral Imaging, Inc. (ASI) SpectraCube system.

FIG. 2 is the graphic output of the Spectral characteristics of the SKY-1 filter set providing multi-band fluorescence excitation.

FIG. 3 shows the excitation and emission spectra of the five dyes commonly used in SKY and SIm.

FIG. 4 is a photograph of the PFGE separation of yeast chromosomes and yeast artificial chromosomes (YACs). Clone 945B6 was selected for the production of the chromosome 18-specific DNA probe. We pick four colonies and ran them sided by side on a low melting point agarose gel. Bands were excised as indicated (1 . . . 3). The expected YAC size (1400 kb) was obtained from the CEPH megaYAC database at the Whitehead Institute for Genome Research. The sizes of bands in the sizemarker lane (szm) are indicated to the right.

FIG. 5 is a photograph of the gel showing the size distribution of DOP-PCR Products. The gel shows the DOP-PCR products after amplification with the oligonucleotide JUN15. Samples FP76 and FP77 were prepared from the DOP-PCR products of YAC clone 945B6 bands 1 and 2, respectively. The size distribution of PCR products ranges from about 100 bp to 600 bp. The sizemarker lane (100 bp) contains 200 ng of a 100 bp DNa ladder. The location of the 500 bp sizemarker band is indicated on the light.

FIG. 6 is a photograph of the gel showing the size distribution of PCR products for the KpnI Family from Chromosome 15. The gel shows the PCR products after amplification with the oligonucleotides KpnI-F1 and -R1.

FIG. 7 shows the fluorescent in situ hybridization (FISH) analysis of human failed-fertilized metaphase II eggs using specific probes for chromosomes 1 (red signals), 16 (yellow signals), 18 (blue signals), and 21 (green signals). Hybridization of the four-probe set to a spread prepared from a normal oocyte (A,B) or an abnormal cell containing an extra chromatid derived from chromosome 1 (‘1’) in addition to the normal bivalents (C,D). Chromosomes are indicated by arrows with numbers. DAPI images are shown in A and C; FISH results are displayed in (B) and (D). The SKY analysis of a freshly spread oocyte involves acquisition of a DAPI image (E), recording a spectral image and displaying it as a false-color RGB image (F), and generation of a karyotyping table that aligns the DAPI, classification color, and false-color RGB images for each chromosome type (G). The eight-probe set was initially tested on metaphase spreads prepared from short-term lymphocyte cultures. The pictures in (H) show the DAPI image of a metaphase spread, in (I) a superposition of the chromosome 18-specific blue fluorescent signals with red fluorescent signal recorded in the red fluorescence channel is shown, and in (J), a superimposition of the chromosome 21-specific green signals on the infrared fluorescent signals delineating targets on chromosomes 15, 16, and Y is shown. Analysis of oocytes and corresponding 1PBs using the eight probe set and SIm detection are shown in K and L.

FIG. 8 is a photograph of two Y chromosome-specific hybridization probes. DNA was selected isolated from a Y-specific BAC clone to compare different labeling strategies. The probe in (8A, 8B) was prepared by enzymatic incorporation of Alexa Fluor 594-dUTP. This reporter fluoresces in the yellow wavelength interval and signals are shown here in red. The probe in (8C) was produced by conjugation of AA-dUTP labeled DNA with DEAC. This very bright probe emits strong fluorescence in a wavelength interval comparable to Spectrum Aqua (Vysis). The white arrows point to the probe signal.

### BRIEF DESCRIPTION OF THE SEQUENCES

SEQ ID NOs: 1-4 are primer sequences.

### DETAILED DESCRIPTION OF THE PREFERRED EMBODIMENT

Present FISH technology allows the analysis of no more than 5 different chromosomes per hybridization. In contrast to present FISH technology, which allows the analysis of only 5 chromosomes per hybridization, herein it is described a first subset of 8 chromosome-specific probes to analyze single cells which can be used to test for abnormalities in chromosomes X, Y, 13, 15, 16, 18, 21 and 22. Herein also is described a full set of 24 chromosome-specific probes to analyze single cells or cell organelles to test for abnormalities in all 24 human chromosomes, wherein the fall set comprises three subsets of 8-chromosome-specific probes to analyze single cells and cell organelles.

These uniquely labeled probes will target all 24 chromosomes including those associated with risk of aneuploidal offspring, such as X, Y, 13, 18, and 21, plus autosomes commonly found in chromosomally abnormal cleavage-stage embryos and spontaneous abortions (Jobanputra et al. 2002), i.e., chromosomes 15, 16 and 22. Single cells or organelles which can be used for such hybridization analysis include but are not limited to, blastomeres, oocytes, polar bodies, spermatagonia, spermatocytes, somatic cells, interphase cells, and tumor cells. In one preferred embodiment, first polar bodies (1 Pbs) or second polar bodies are biopsied from a single oocyte and undergo analysis with the presently described probe sets.

In one embodiment, the first subset of 8 chromosome-specific probes to analyze 1 Pbs can also be used in the full set of probes, wherein the full set comprises three subsets of 8-chromosome-specific probes.

As used herein, a “set” of probes refers to a set, panel or subset of 8, 16 or 24 chromosome specific probes. In a preferred embodiment, a “full set” of 24 probes is comprised of 3 subsets of chromosome specific probes, wherein each subset comprises a set of 8 chromosome specific probes. Thus, a “subset” of 8 chromosome specific probes is to be defined as the equivalent of “a set of 8 chromosome specific probes.”

In another embodiment, a method of fluorescence in situ hybridization (FISH) to accurately determine the number and type of all human chromosomes in interphase cells and first polar bodies biopsied from non-inseminated oocytes. The assay is based on sequential hybridization of three subsets of chromosome-specific DNA probes with each subset comprised of 8 different probes. Also described are methods of selection or generation of suitable probe DNAs, non-isotopical labeling of DNA with fluorescent haptens and optimization of hybridization protocols.

Probe Set and Selection of Probes

In a preferred embodiment, FISH probes are used from the collection of probes prepared earlier as described in Weier et al., “Chromosome abnormalities in human arrested preimplantation embryos: a multiple-probe FISH study,” Am J Hum Genet. 1994 July; 55(1):150-9, 1994; Munne et al., “Assessment of numeric abnormalities of X, Y, 18, and 16 chromosomes in preimplantation human embryos before transfer,” Am J Obstet Gynecol. 1995 April; 172(4 Pt 1):1191-9; discussion 1199-201, 1995; Fung, J., H. U. G. Weier, J. D. Goldberg, R. A. Pedersen (1999) Simultaneous scoring of 10 chromosomes (9, 13, 14, 15, 16, 18, 21, 22, X, Y) in interphase nuclei by using Spectral Imaging, Proc. of SPIE 3604:218-226; Zitzelsberger H F, O'Brien B, Weier HUG (2002) Multicolor FISH techniques for the detection of inter- and intrachromosomal rearrangements. In: FISH Technology. B. Rautenstrauss and T. Liehr (Eds.), Springer Verlag, Heidelberg, pp. 408-424, which are hereby incorporated by reference.

Specific probes and their preparation that may be used in the probe sets of the invention include those described by Weier, H.-U., Kleine, H.-D., Gray, J. W. (1991) Labeling of the centromeric region on human chromosome 8 by in situ hybridization. Human Genetics 87:489-494 and Weier, H.-U., Rosette, C.D., Matsuta, M., Zitzelsberger, H., Matsuta, M., Gray, J. (1994) Generation of highly specific DNA hybridization probes for chromosome enumeration in human interphase cell nuclei: isolation and enzymatic synthesis of alpha satellite DNA probes for chromosome 10 by primer directed DNA amplification, Meth Mol Cell Biol 4:231-248, along with those described in the References listed herein, which are herein incorporated by reference.

In a preferred embodiment, the probes listed hi Table 2 or 3 are used in detecting each of the 24 human chromosomes as specified. All GenBank and GDB sequences listed in the Sequence Listing are hereby incorporated by reference in their entirety. The probes listed in Tables 2 and 3 are meant to be exemplary and should not be considered as limiting the invention. One of skill in the art could select other probes to the target chromosomes.

In a preferred embodiment, other probes can also be developed according to known procedures in the art, briefly described herein. Methods of preparing probes are well known to those of skill in the art (see, e.g. Sambrook et al., Molecular Cloning: A Laboratory Manual (2nd ed.), Vols. 1-3, Cold Spring Harbor Laboratory, (1989) or Current Protocols in Molecular Biology, F. Ausubel et al., ed. Greene Publishing and Wiley-Interscience, New York (1987)), which are hereby incorporated by reference.

Another example of a method that can be used to develop probes for the present hybridization analysis is found in U.S. Pat. Nos. 5,427,932 and 5,888,730, and Cassel, M. J., Munné, S., Fung, J., Weier, H.-U. G. (1997), Carrier-specific breakpoint-spanning DNA probes for pre-implantation genetic diagnosis [PGD] in interphase cells. Hum Reprod 12:101-109, all of which are hereby incorporated by reference. The method as applied to development of probes for determination of chromosomal abnormality can be as follows. The probes are most easily prepared by combining and labeling as described herein.

Prior to use, larger constructs can be fragmented to provide smaller nucleic acid fragments that easily penetrate the cell and hybridize to the target nucleic acid. Fragmentation can be by any of a number of methods well known to those of skill in the art, including random priming, nick translation, and tailing. Treatment of larger size probes include sonication, or enzymatic restriction to selectively cleave the molecule. Probes are preferably fragmented to or are made with an average fragment length ranging from about 50 bp to about 2000 bp, more preferably from about 100 bp to about 1000 bp and most preferably from about 150 bp to about 500 bp.

Preferred probes include DNA double-stranded probes, which may require denaturation, alkaline treatment or exonuclease digestion, single-stranded DNA probes and oligonucleotides, RNA probes or peptide nucleic acid (PNA) probes. All DNA and RNA probes can be prepared by nick translation or random priming with commercial kits (such as BIOPRIME, BIONICK available from Invitrogen). Synthetic oligonucleotide probes can prepared and obtained commercially. Methods of making and using PNA probes are described in Peter E. Nielsen, ed., Peptide Nucleic Acids: Protocols and Applications (Second Edition), Horizon Bioscience, The Panum Institute, Copenhagen, January 2004, hereby incorporated by reference.

Each set of probes should be prepared with similar hybridization parameters and blocking requirements. In general, single copy probes like those prepared from BAC or YAC clones require blocking of interspersed repeat (LINEs, SINEs), which is commonly achieved by addition of unlabelled COT1 (Invitrogen) DNA. The COT1 DNA contains just the highly repeated DNA sequences such as SINEs, LINEs, ALUs and satellite DNA. In some applications it is necessary to block the hybridization capacity of repetitive sequences. In one embodiment, human genomic DNA is used as an agent to block such hybridization. The preferred size range is from about 200 bp to about 1000 bases, more preferably between about 400 to about 800 bp for double stranded, nick translated nucleic acids.

Probes that target DNA repeats, on the other hand, can often be prepared highly specifically and do need minimal or no blocking prior to or during hybridization. Thus, single copy and DNA repeat probes are best used separately. It would be preferred that the hybridization strategy be applied to a set of 8 locus-specific bacterial artificial chromosome (BAC) or yeast artificial chromosome (YAC) probes first because BAC probes target 100-300 kb and target sizes for YAC derived probes can exceed 1 megabase. Clones are selected that produce tightly localized hybridization domains in interphase cells, which will be easy to score. In one embodiment, after clone selection, hybridizations are performed in the presence or absence of human COT1 blocking DNA to estimate the amount of cross-hybridization caused by various type of DNA repeats in the single copy YAC probes.

The selection of probe clones can be guided by quite a number of rules. Examples for such rules are the inclusion/exclusion of probes for highly repeated DNA targets. Some of these probes bind to DNA targets (pericentromeric heterochromatin, satellite DNA, etc.) that are heteromorph. Thus, an individual might carry one chromosome with a large target leading to a strong signal, while the other homologue carries a much smaller repeat cluster. In extreme, but not very rare cases, the difference might be so drastic that (especially when the hybridization efficiency is compromised) only the strong signal is scored by the observer. Thus, to understand the consequences of using heteromorphic FISH targets one has to keep in mind that in many of the clinical investigations only a single first polar body or cell will be available for analysis. So, in some rare occasions, the observer could be unable to judge whether missing a signal is the result of a missing chromosome or an extreme case of heteromorphism.

Using the above strategy, a set of uniquely labeled, highly specific DNA probes for chromosomes 13, 15, 16, 18, 21, 22, X and Y was selected and defined as “Set 1.” Set 1, shown in Table 6, was found to be suitable to score chromosomes in first polar bodies, oocytes, interphase and metaphase cells. Table 6 below lists the probes and labeling scheme for Set 1. The hybridization efficiencies of individual probes or probe combinations were determined.

Some of the DNA probes that were used for Set 1 were selected from heteromorphic regions. One example is the novel satellite II DNA probe specific for chromosome 15 based on human DI 5Z1 that was used in Set 1. This probe has the unique feature that it can be tailored to either score copies of chromosomes 15 or detect heteromorphisms involving the locus DI 5Z1. Because this probe targets a heteromorphic region, different signal intensities for the different homologues can sometimes be observed. The use of BAC probes for single copy targets on the long arm of chromosome 15 was also investigated, but the use of BAC probes in commercial applications might lead to complicated licensing issues for the user. Another probe that has been given a closer look was the alpha satellite probe for chromosome 18 (target D18Z1) contained in Set 1. However, looking at probe performance in this project as well as other applications, no problem was found with this probe.

Alternatively, probes can be produced by amplifying (e.g. via PCR) selected subsequences from the amplicons disclosed herein in Table 2 and Table 3. The sequences provided herein permit one of skill to select primers that amplify sequences from one or more exons located within the target regions of each chromosome.

Labeling Strategies

Any method of imaging may be used to detect the chromosomal make-up of single cells may be used. Methods of labeling nucleic acids are well known to those of skill in the art. Preferred labels are those that are suitable for use in in situ hybridization. The nucleic acid probes may be detectably labeled prior to the hybridization reaction. Alternatively, a detectable label which binds to the hybridization product may be used. Such detectable labels include any material having a detectable physical or chemical property and have been well-developed in the field of in immunoassays.

As used herein, a “label” is any composition detectable by spectroscopic, photochemical, biochemical, immunochemical, or chemical means. Useful labels in the present invention include radioactive labels (e.g., ³²P, ¹²⁵I, ¹⁴C, ³H, and ³⁵S), fluorescent dyes (e.g. fluorescein, rhodamine, Texas Red, etc.), electron-dense reagents (e.g. gold), enzymes (as commonly used in an ELISA), calorimetric labels (e.g. colloidal gold), magnetic labels (e.g. DYNABEADS™), and the like. Examples of labels which are not directly detected but are detected through the use of directly detectable label include biotin and dioxigenin as well as haptens and proteins for which labeled antisera or monoclonal antibodies are available.

The particular label used is not critical to the present invention, so long as it does not interfere with the in situ hybridization of the stain. However, stains directly labeled with fluorescent labels (e.g. fluorescein-12-dUTP, Texas Red-5-dUTP, etc.) are preferred for chromosome hybridization

A direct labeled probe, as used herein, is a probe to which a detectable label is attached. Because the direct label is already attached to the probe, no subsequent steps are required to associate the probe with the detectable label. In contrast, an indirect labeled probe is one which bears a moiety to which a detectable label is subsequently bound, typically after the probe is hybridized with the target nucleic acid.

In addition the label should be detectable in as low copy number as possible thereby maximizing the sensitivity of the assay and yet be detectible above any background signal. Finally, a label must be chosen that provides a highly localized signal thereby providing a high degree of spatial resolution when physically mapping the stain against the chromosome. Particularly preferred fluorescent labels include fluorescein-12-dUTP and Texas Red-5-dUTP.

The labels may be coupled to the probes in a variety of means known to those of skill in the art. In a preferred embodiment the nucleic acid probes will be labeled using nick translation or random primer extension (Rigby, et al. J. Mol. Biol., 113: 237 (1977) or Sambrook, et al., Molecular Cloning—A Laboratory Manual, Cold Spring Harbor Laboratory, Cold Spring Harbor, N.Y. (1985)).

Labeled nucleoside triphosphates (FITC-12-dUTP, dig.-11-dUTP, Cy3/Cy5-dUTP) can be purchased commercially from, for example, Roche and Amersham respectively. To label DNA probes with Spectrum Aqua (diethyl aminomethyl coumarin, DEAC) or Pacific Blue, one can use commercial kits such as the ARES kit (Molecular Probes, Eugene, Oreg.). This kit allows the amination of DNA by incorporation of aminoallyl-dUTP. These aminogroups then provide the functional groups to couple fluorochromes (Spectrum Aqua, Pacific Blue, Alexa dyes; Molecular Probes) to the DNA.

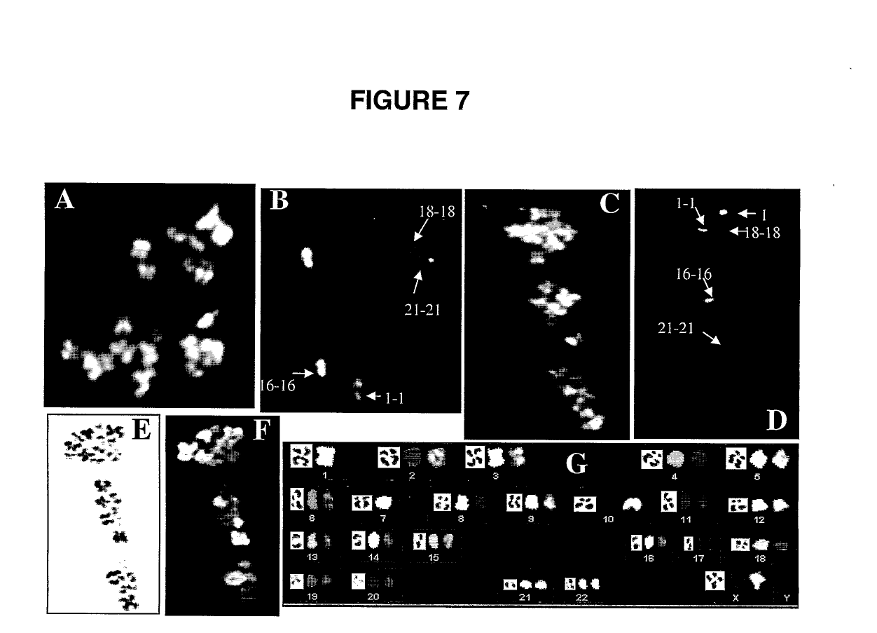

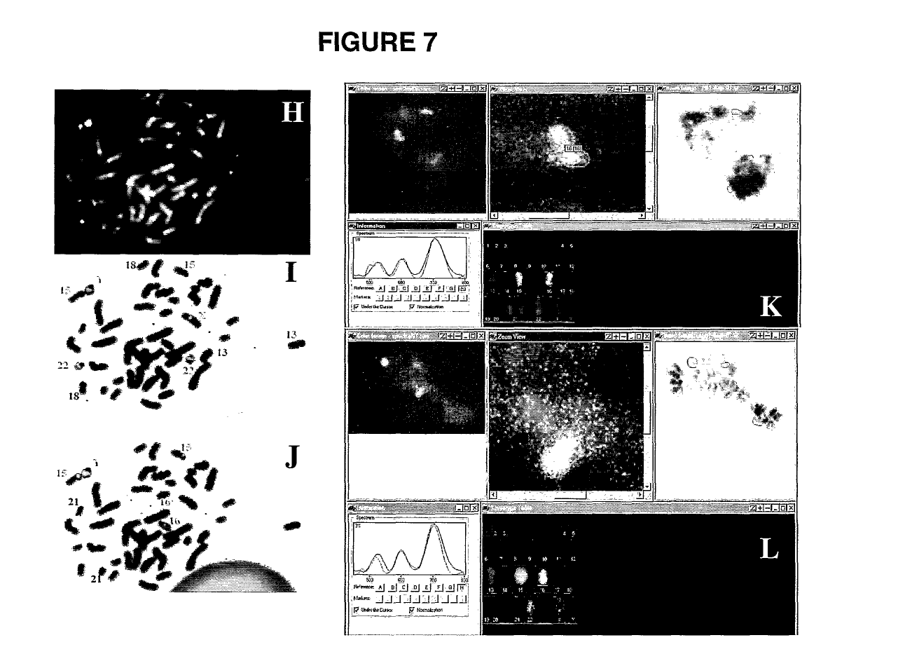

In another embodiment, fluorochrome-labeled probes can be prepared from Pulsed Field Gel electrophoresis (PFGE) purified yeast artificial chromosome (YAC) clones specific for human chromosomes such as, chromosomes 13, 18, 21 or 22. We selected previously cloned DNA repeats for the preparation of blue fluorescent, DEAC-labeled probes for chromosomes 1, 16, X and Y and determined their hybridization strength (FIG. 7). The three probes specific for chromosomes 16, X and Y were then labeled with unique fluorochromes.

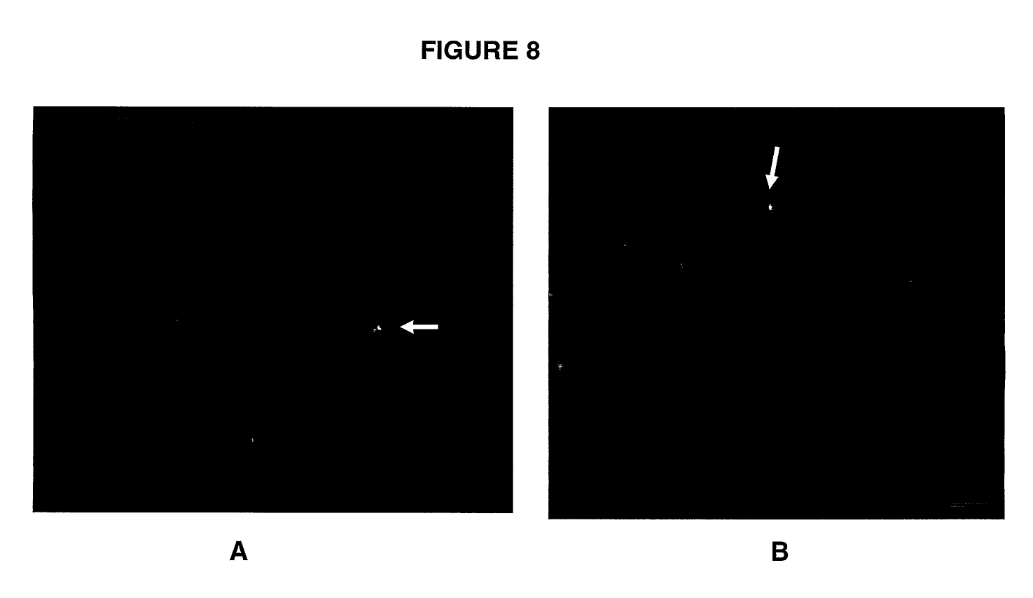

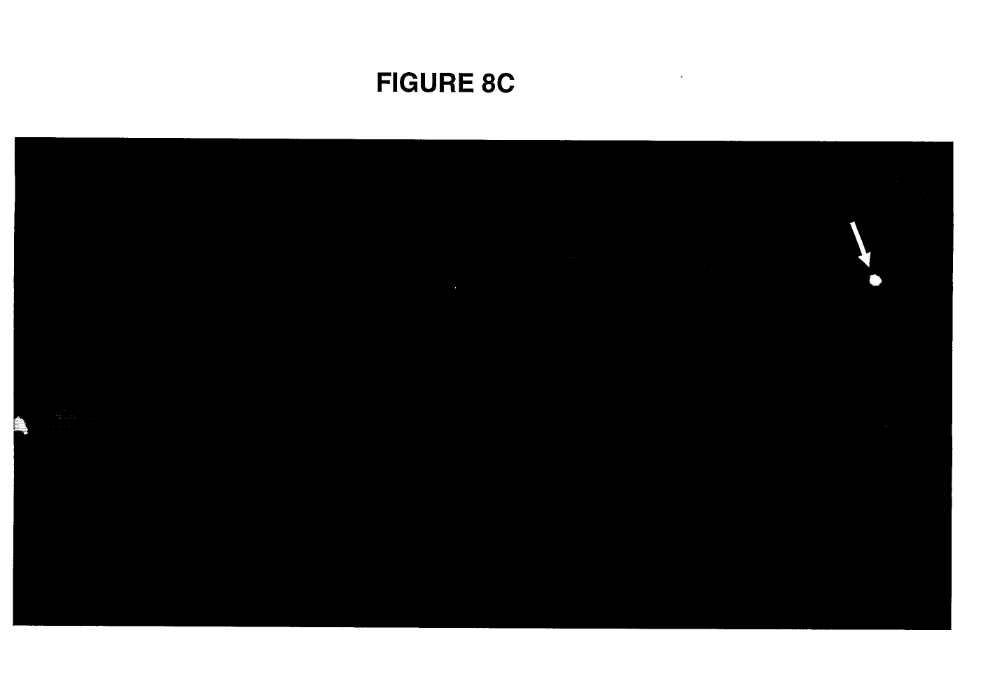

The issues of ‘how’ to label a DNA probe with ‘which’ reporter molecule are almost as important as the proper clone selection. Regarding preferred probe labels, various labeling protocols were evaluated that matched the commercially available fluorescent reporters and microscope filters available. The use of 3′-tailing was investigated using terminal deoxynucleotidyl transferase (TdT), PCR labeling, random priming, nick translation and chemical modification of modified (aminated) DNA. Briefly, 3′-tailing showed the worst performance leading to faint probes. PCR labeling seemed working well for small DNA repeat probes, but it is costly due to inefficient use of expensive fluorochrome-conjugated dNTPs. Nick translation and incorporation of fluorescent dUTP gave probes bright enough to be detected by eye in the microscope, but in general weaker than random primed probes made from the same template DNA (FIGS. 7 and 8). Surprisingly, the incorporation of an aminoallyl-dUTP nucleoside triphosphate (AA-dUTP) via random priming followed by covalent coupling of a fluorescent reporter (such as Invitrogen's/Molecular Probes's Alexa Fluor 594-NHS ester) resulted in probes of unsurpassed brightness and specificity. See FIG. 8C as compared to FIGS. 8A and 8B. As shown in FIG. 8, DNA isolated from a Y chromosome-specific BAC clone (RP11-243E13) was used to compare the labeling strategies. This BAC contains multiple copies of the Y-specific 3.4 kb satellite III repeat and labels essentially the entire long aim of the Y chromosome (FIG. 8C).

Detection of Probes

In a preferred embodiment, abnormalities in a single cell or cellular organelles are detected through the hybridization of three sets of 8 probes to target human chromosomes. Suitable hybridization formats are well known to those of skill in the art and include, but are not limited to, variations of Southern Blots, in situ hybridization and quantitative amplification methods such as quantitative PCR (see, e.g. Sambrook, supra., Kallioniemi et al., Proc. Natl Acad Sci USA, 89: 5321-5325 (1992), and PCR Protocols, A Guide to Methods and Applications, Innis et al., Academic Press, Inc. N.Y., (1990)).

In a preferred embodiment, the 24 human chromosomes are identified using in situ hybridization. Generally, in situ hybridization comprises the following major steps: (1) isolating and fixation of the single cell or biological structure containing the target chromosomes to analyzed; (2) prehybridization treatment of the cell or biological structure to increase accessibility of target DNA, and to reduce nonspecific binding; (3) hybridization of the mixture of probes to the target chromosomes in the biological structure or cell; (4) posthybridization washes to remove unbound probes and posthybridization treatment such as washes, blocking, detection and amplification; and (5) detection of the hybridized probes. The reagent used in each of these steps and their conditions for use may vary depending on the particular application.

For example, fixation of the single cell can include the steps of first immersing the cell in a drop of hypotonic solution, e.g., 75 mM KCl, to allow the cell to swell and cover a larger area. A fixative, such as acetic acid:methanol 1:3 v/v, can be pipetted onto the cell to hydrolyze proteins, remove hypotoric solution, and fix the cell to untreated glass slides. Treatment with RNAase is also recommended before or after fixation. Other post-fixation treatments can include enzymatic pretreatments, post fixation washes in buffers of different salt concentration, secondary fixation using paraform aldehyde or acetone for example, and incubation at elevated temperature (e.g. in ambient temperature in air for 2 weeks or 80° C. for 1-2 hours) which acts as a method of aging to retain DNA throughout further denaturation and hybridization steps.

Prior to contacting the probes with the single cell or the organelles to hybridize the probes, a denaturation step can be performed thermally or chemically, but is only necessary if DNA double-stranded (ds) probes are selected. After hybridization of the probes to the target chromosomes in the single cell or organelle, any number of posthybridization treatments can occur. These can include, but are not limited to, posthybridization washes to remove unbound probes and post hybridization processing such as washes, blocking, detection and amplification. Finally detection of the hybridized probes can be carried out. In a preferred embodiment, the detection of the probes is performed using a filter-based fluorescent microscope, optionally equipped with a spectral imaging system.

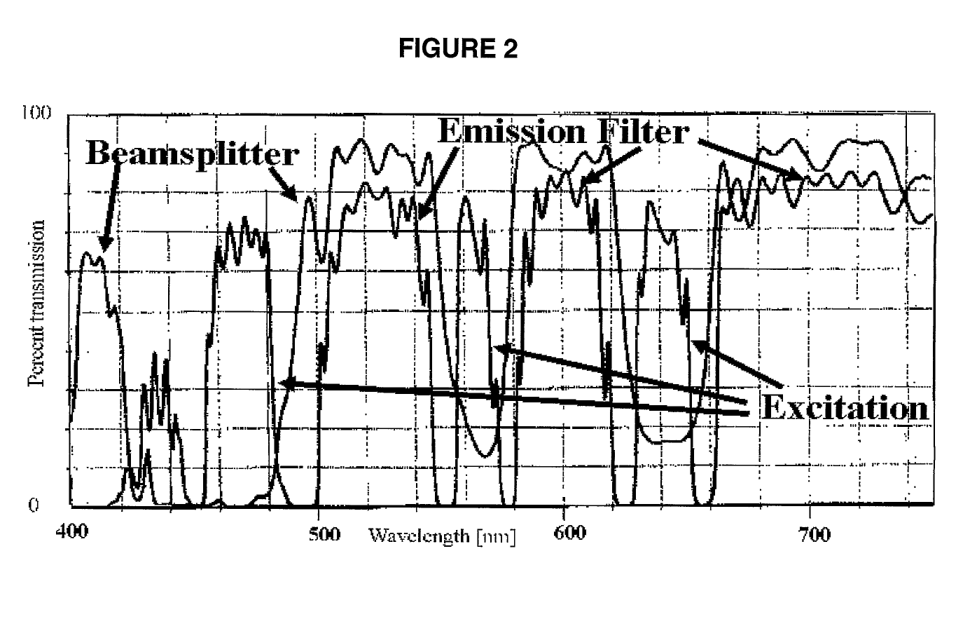

Filter-Based Fluorescent Microscope. In a preferred embodiment, the probes selected are fluorescent probes thereby allowing the detection of the hybridized probes using a filter-based fluorescent microscope. In a preferred embodiment, the microscope is equipped with a CCD camera and fluorescent filters, such as FITC or Texas Red filters, for fluorochrome excitation and observation. In a preferred embodiment, the multiple band pass filter set (ChromaTechnology, Brattleboro, Vt.) (FIG. 2) can be used for fluorochrome excitation to provide three broad emission bands centered around 470 nm, 565 nm and 640 nm. Fluorescence can be recorded through a multi-bandpass filter with broad transmission peaks in the vicinities of 520 nm (green), 600 nm (red) and 700 nm (infrared, FIG. 2) (Schroeck et al., 1996) to match the excitation/emission profile of the selected fluorochromes. In one embodiment, the microscope has a combination of eight excitation/emission filters for the eight dyes used for each 8-probe set such as DAPI, Spectrum Aqua or Pacific Blue and the CCD camera.

One detection strategy is based on interchangeable excitation and fluorescence emission filters termed multi-fluor FISH or mFISH which is described by Speicher M R, Gwyn Ballard S, Ward D C. Karyotyping human chromosomes by combinatorial multi-fluor FISH. Nat. Genet. 1996 April; 12(4):368-75, which is hereby incorporated by reference.

Filter-Based Fluorescent Microscope with Spectral Imaging (SIm) system. In another preferred embodiment, the hybridized fluorescent chromosome-specific probes are detected using a filter-based fluorescent microscope with a spectral imaging system. A recent development in fluorescence microscopy termed ‘Spectral Imaging (SIm)’ now allows the recording of an entire spectrum from a fluorescent object with high resolution. Existing SIm instrumentation can record fluorescence spectra from 400 nm to 1100 nm with about 10 nm resolution and has been described by Schröck et al., (1996) Multicolour spectral karyotyping of human chromosomes. Science 273: 494-497; Liyanage et al., Multicolour spectral karyotyping of mouse chromosomes, Nat. Genet. 1996 November; 14(3):312-5 1996; Garini et al., Spectral karyotyping. Bioimaging 4, 65-72 (1996); Fung et al., Spectral imaging in preconception/preimplantation genetic diagnosis of aneuploidy: multicolor, multichromosome screening of single cells, J Assist Reprod Genet. 1998 May; 15(5):323-30, which are hereby incorporated by reference. In a preferred embodiment, the spatial resolution, limited by the diffraction in the light microscope, is typically better than 500 nm.

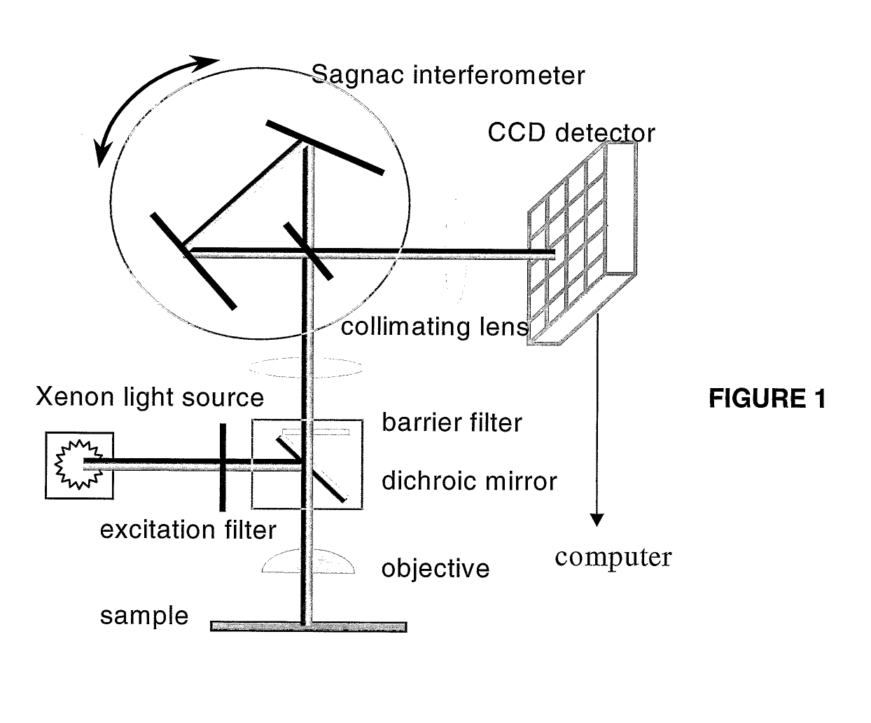

Typically, SIm combines the techniques of fluorescence microscopy, charge-coupled device (CCD) camera and Fourier spectroscopy (FIG. 1). The light emitted from each point of the sample is collected with the microscope objective and sent to a collimating lens. The collimated light travels through an optical head (interferometer) and is focused on a charged coupled device (CCD). The data are collected and processed with a personal computer. The interferometer divides each incoming beam (the light projected from the microscope) into two coherent beams and creates a variable optical path difference (OPD) between them. The beams are then combined to interfere with each other, and the resulting interference intensity is measured by the CCD detector as a function of the OPD. The intensity vs. OPD is called ‘an interferogram’. The spectrum, i.e., intensity as function of wavelength, can be recovered from the interferogram by a relatively simple mathematical operation called ‘Fourier transformation’. This transformation is performed in the personal computer attached to the Spectral Imaging system. The spectral resolution depends on the number of interferometric steps. For most experiments, a resolution of 10-20 nm (equivalent to 64-128 steps) is sufficient.

First applications of SIm, also termed ‘Spectral Karyotyping (SKY),’ screened metaphase spreads for translocations. See Schlöck et al. 1996; Zitzelsberger et al., Cytogenetic changes in radiation-induced tumors of the thyroid, Cancer Res. 1999 Jan. 1; 59(1):135-40; Zitzelsberger et al., Clonal chromosomal aberrations in simian virus 40-transfected human thyroid cells and in derived tumors developed after in vitro irradiation, Int J Cancer. 2001 Jun. 20; 96(3):166-77;. Weier, H. U. G., S. Munné, R. A. Lersch, C. Marquez, J. Wu, R. A. Pedersen, J. Fung. (1999) High performance analysis of single interphase cells with custom DNA probes spanning translocation breakpoints. Proc. of SPIE 3604: 227-236; Fung, J., H. U. G. Weier, J. D. Goldberg, R. A. Pedersen (1999) Simultaneous scoring of 10 chromosomes (9, 13, 14, 15, 16, 18, 21, 22, X, Y) in interphase nuclei by using Spectral Imaging. Proc. of SPIE 3604:218-226.

Based on these first applications, a preferred embodiment allows hybridization of the human chromosomes with 24 chromosome-specific whole chromosome painting (WCP) probes labeled individually with Spectrum Green, Spectrum Orange, Texas Red, Cy5, or Cy5.5 and combinations thereof, for rapid analysis of metaphase spreads in a single experiment.

In a preferred embodiment, using a Xenon light source, the spectral image is generated by acquiring 80-130 interferometric frames per object. The sample emission spectra (400-850 nm) can be measured simultaneously at all points in the microscopic image. The spectral information is displayed by assigning specific colors, e.g., red, green or blue, to certain ranges of the spectrum. This display, e.g., an RGB display, renders chromosomes that were labeled with spectrally overlapping fluorochromes or fluorochrome combinations in a similar color. Based on the measurement of the pure spectrum for each chromosome a spectral classification algorithm is applied to allow the assignment of a pseudo-color to all pixels in the image that have the same fluorescence spectrum. Chromosome identification is then performed by comparison of the measured spectra with pre-recorded reference spectra, and chromosomes are displayed in ‘classification’ colors to facilitate the detection of translocations involving non-homologous chromosomes in SKY analyses of metaphase spreads or the loss of chromosomes or detection of extra chromosomes in SIm analysis of interphase cells. See J. Fung et al., Multilocus genetic analysis of single interphase cells by spectral imaging, Hum Genet. 2000 December; 107(6):615-22 hereby incorporated by reference.

In a preferred embodiment, the fluorescence spectra of the reporter molecules should have minimal overlap. In other embodiment, the spectra of the reporter molecules may be partially overlapping, as spectral overlap can be resolved by ‘Spectral Un-Mixing (SUN)’ (developed by ASI; see also Macville M V et al., Spectral imaging of multi-color chromogenic dyes in pathological specimens. Anal Cell Pathol. 2001; 22(3):133-42). Images are imported and analyzed after being recorded with either the Spectral imaging system or a combination of excitation/emission filters for dyes such as DAPI, Spectrum Aqua or Pacific Blue and the CCD camera.

In a preferred embodiment, chromosomal make-up of single cells or cell organelles are detected using the spectral imaging methods (SIm) as described above, because of the high spectral resolution of SIm. As mentioned above, the ratio-labeling color scheme applied for SKY will not work for the determination of the intracellular number of chromosomes, because the different hybridization targets could spatially overlap. Therefore, in a preferred embodiment, each DNA probe is labeled with a unique reporter molecule. In one embodiment, the reporter molecule is a fluorochrome that is commercially available. Their emission maxima should be spaced sufficiently to allow discrimination by a Spectral Imaging or filter based system. For example, Table 1 shows a preferred scheme to uniquely label eight chromosome-specific hybridization targets and counterstain genomic DNA and having their emission maxima spaced sufficiently to allow discrimination by a Spectral Imaging system.

TABLE 1
Nine-color, eight target spectral analysis

| | Primary | Secondary | Excitation | Emission |
|---|---|---|---|---|
| Target | Label | Label | (nm) | (nm) |
| Genomic DNA | DAPI | — | 363 | 461 |
| | (Marina Blue) | | (365) | (460) |
| Target 1 | Pacific Blue | — | 410 | 455 |
| Target 2 | Spectrum Aqua | — | 436 | 480 |
| Target 3 | FITC | — | 495 | 528 |
| Target 4 | Cy3 | — | 552 | 565 |
| Target 5 | Biotin | Cy3.5 | 581 | 596 |
| Target 6 | Texas Red | — | 596 | 620 |
| Target 7 | Cy5 | — | 650 | 667 |
| Target 8 | Digoxigenin | Cy5.5 | 678 | 703 |

It was further demonstrated that Spectral Imaging (SIm) analysis can accurately classify eight different DNA probes based on their fluorescence spectra and signal intensity, and that several sets of probes can be applied in repeated hybridizations. In a preferred embodiment, the proposed labeling scheme for 24 probes targeting all 24 human chromosomes, clones selected and the type of their respective targets are as shown in Table 2. In addition to Set 1, herein described is the selection of DNA probes for the remaining 16 chromosomes and development of a DNA labeling scheme that allows the detection of bound probes in a filter-based fluorescence microscope. Table 2 defines a preferred composition of Sets 2 and 3, to investigate the specificity, signal strength and compatibility of these probes. The sequences and accession records for the cited NCBI GenBank Accession sequences are incorporated by reference. The probe suggested for chromosome 1 under GDB:157592 was disclosed by Buroker N, et al. A hypervariable repeated sequence on human chromosome 1p36, Hum Genet. 1987 October; 77(2):175-81. It can be ordered commercially from Qbiogene (Carlsbad, Calif.) under catalog #PONC0136 as 1p36 Midi-Satellite Probe, or ordered from American Type Culture Collection (ATCC) under ATCC No. 40323, 59862, or 59863.

TABLE 2
Three DNA probe sets, hybridization targets, labeling schemes and probe clones.
Chromosome

| Set 1 | 13 | 15 | 16 | 18 | 21 | 22 | X | Y |
|---|---|---|---|---|---|---|---|---|
| color | red | aqua | infrared | orange | green | gold | infrared | blue |
| label | Alexa 594 | DEAC | Cy5.5 | Cy3 | Alexa 488 | Alexa 532 | Cy5 | Pacific Blue |
| Genbank ID | Z24331 | M30706 | X06138 | M65181 | X52289 | G11775 | X02418 | AC068123 |
| clone/DNA | YAC 900G6 | PCR 8509 | pHuR195 | pYAM9-60 | YAC 141G6 | YAC 849E9 | 868TA-3 (3) | RP11-242E13 |

| Set 2 | 14 | 10 | 19 | 17 | 20 | 11 | 12 | 9 |
|---|---|---|---|---|---|---|---|---|
| color | red | aqua | infrared | orange | green | gold | infrared | blue |
| label | Alexa 594 | DEAC | Cy5.5 | Cy3 | Alexa 488 | Alexa 532 | Cy5 | Pacific Blue |
| Genbank ID | Z16978 | X63622 | CL423288 | AC131274 | G06987 | M21452 | G03348 | X06137 |
| clone/DNA | YAC 886A3 | 609/51 (3) | RP11-197O4 | RP11-285M22 | YAC 858D9 | 238′-5′ (3) | YAC 770C7 | PCR 1131 (3) |

| Set 3 | 3 | 5 | 8 | 4 | 2 | 6 | 7 | 1 |
|---|---|---|---|---|---|---|---|---|
| color | red | aqua | infrared | orange | green | gold | infrared | blue |
| label | Alexa 594 | DEAC | Cy5.5 | Cy3 | Alexa 488 | Alexa 532 | Cy5 | Pacific Blue |
| Genbank ID | Z51343 | NT_034772 | M64779 | G04091 | AC073415 | G04505 | AJ295152 | GDB: 157592 |
| clone/DNA | YAC 958B5 | RP11-182C19 | 8B/9 (3) | YAC 887E8 | RP11-84C2 | YAC 819A10 | 680TA-4 (3) | p1-79 |

(1) All YAC clones are from the CEPH/Genethon library, Paris, France (Cohen D,
Chumakov I, Weissenbach J., First generation of the physical map of the human genome, C R Acad Sci III. 1993 December; 316(12): 1484-1488.) 'PCR' refers to PCR reaction numbers in the Weier lab.
(2) RP11 BAC clones are from Roswell Park Cancer Institute, Invitrogen cat. #RPCI11.C.
(3) These DNA repeat probes were prepared in-house and can be replaced with those probes commercially available from Vysis, Inc., Downers Grove, IL; Qbiogene, Carlsbad, CA; Cambio, Cambridge UK; Biological Industries, Kibbutz Beit Haemek, Israel, or other suppliers.
(4) The probe suggested for Chromosome 1 under GDB: 157592 can be found at The GDB Human Genome Database which is the official world-wide database for the annotation of the Human Genome, found at URL<http://www.gdb.org>.

In another preferred embodiment, the frill set of 24 chromosome-specific probes, the hybridization targets and chromosomal position and band, labeling schemes and probe clones are described in Table 3. Each probe is called out and comprised of the clone sequence found under the GenBank Accession numbers or GDB ID number provided and/or the end sequences are used to generate the probe sequence in the clones identified. The table lists comprehensively the chromosome number, band, and position targeted, the probe name, label, and probe type. Each of the clones, sequences and records in the GenBank Accession Numbers are hereby incorporated by reference.

In the course of probe development using BAC probes, it was noticed that organizations such as the Roswell Park Cancer Institute (RPCI) or the Children's Hospital of Oakland Research Institute (CHORI) are carefully watching the use of clones generated under their auspices. While academic use is typically allowed royalty free, the commercial use of any clone or its derivatives might require a license. Thus proper selection of probe clones might accelerate the process of bringing the probe sets to the market by expediting the negotiation processes.

It is further contemplated that the probe set can be optimized with regard to probe specificity, signal strength, ease of use and cost, according to the teachings of the invention and using methods known in the art.

Applications

Chromosomal Abnormality. Iii a preferred embodiment, the three sets of 8-probe sets are used for analysis to determine chromosomal abnormality prior to fertilization or implantation during the course of in vitro fertilization. In one embodiment, the method provides for analysis of all 24 human chromosomes including those associated with risk of aneuploid offspring, such as X, Y, 13, 18, and 21, plus autosomes commonly found in chromosomally abnormal cleavage-stage embryos and spontaneous abortions, i.e., chromosomes 15, 16 and 22.

The method further provides for analysis of a single cell or cellular organelle. In a preferred embodiment, the single cells or cell organelles analyzed for such hybridization analysis are oocytes, blastomeres, polar bodies, spermatocytes, somatic cells, interphase cells, and tumor cells.

Prior to fertilization and implantation, analysis of polar bodies biopsied from unfertilized oocytes or zygotes or the analysis of a single blastomere removed from a 6- to 8-cell stage human embryo using the presently described sets of hybridization probes, wherein detection is carried out preferably using spectral imaging as described. Since only those eggs (or embryos) considered chromosomally normal are replaced to women undergoing in vitro fertilization in order to reduce embryo wastage and prevent trisomic offspring, the present hybridization sets and methods will provide a rapid and innovative tool for chromosome enumeration and analysis.

In another embodiment, a method of fluorescence in situ hybridization (FISH) to accurately determine the number and type of all human chromosomes in interphase cells and first polar bodies biopsied from non-inseminated oocytes using the full set of three panels of 8-chromosome-specific probes described in Table 2 or 3. The assay is based on sequential hybridization of three sets of chromosome-specific DNA probes with each set comprised of 8 different probes. Also described are methods of selection or generation of suitable probe DNAs, non-isotopical labeling of DNA with fluorescent haptens and optimization of hybridization protocols.

In one embodiment, the full set of probes described in Table 2 or 3 is used to determine the chromosome-specific rates of aneuploid cells for various age groups as described in Examples 3 and 4.

In another embodiment, the full set of probes is used to check the rate of aneuploidy in the reproductive cells of a patient who has undergone chemotherapy. For example, the spermatocytes of young men of reproductive age who have undergone chemotherapy can be assessed by the full set of probes.

In another embodiment, the full set of probes described in Table 2 or 3 is used to analyze tumor cells for chromosomal abnormality. In a preferred embodiment, the tumor cell is biopsied from a cancer patient. It is known that many leukemias, lymphomas, myelomas, sarcomas, breast cancers, prostate and ovarian cancers, exhibit chromosomal abnormalities such as translocation, amplification or deletion.

It is further contemplated that at least 24 probes directed to several different target loci or amplicons may be developed and used as three 8-probe sets using the nine-color, eight target scheme in Table 1, to analyze a tumor cell to provide a chromosomal profile of the cancer. For example, at least 24 probes to detect amplification in breast cancer can be labeled using the scheme in Table 1. In another Example, 24 probes selected from known FISH probe libraries (available commercially from companies such as Genzyme Corporation, Cambridge, Mass.) can be labeled using the scheme in Table 1. Detection using SIm can determine if the loci amplification or abnormality is then detected in a tumor sample.

Hybridization protocols for particular tumor applications are described in Pinkel et al. Proc. Natl. Acad. Sci. USA, 85: 9138-9142 (1988) and in EPO Pub. No. 430,402. Suitable hybridization protocols can also be found in Methods in Molecular Biology Vol. 33, In Situ Hybridization Protocols, K. H. A. Choo, ed., Humana Press, Totowa, N.J., (1994). In a particularly preferred embodiment, the hybridization protocol of Kallioniemi et al., Proc Natl Acad Sci USA, 89: 5321-5325 (1992) is used.

The FISH methods for detecting chromosomal abnormalities can be performed on nanogram quantities of the subject nucleic acids. Paraffin embedded tumor sections can be used, as can fresh or frozen material. Because FISH can be applied to the limited material, touch preparations prepared from uncultured primary tumors can also be used (see, e.g., Kallioniemi, A. et al., Cytogenet. Cell Genet. 60: 190-193 (1992)). For instance, small biopsy tissue samples from tumors can be used for touch preparations (see, e.g., Kallioniemi, A. et al., Cytogenet. Cell Genet. 60: 190-193 (1992)). Small numbers of cells obtained from aspiration biopsy or cells in bodily fluids (e.g., blood, urine, sputum and the like) can also be analyzed. For prenatal diagnosis, appropriate samples will include amniotic fluid and the like.

### Example 1

### Probe Preparation

The following example describes procedures to generate chromosome-specific DNA probes suitable for multi-probe/multi-color analysis of first polar bodies (I PBs) and oocytes in the following Examples. Here, we describe the preparation of DNA probes from pulsed field gel electrophoresis (PFGE) purified yeast artificial chromosome (YAC) clones. A novel application of in vitro DNA amplification using the polymerase chain reaction (PCR) to isolate DNA repeats that map to the heterochromatic region of human chromosome 15 is included.

Purification of human YAC DNA. Since the size of the Saccharomyces cerevisiae genome is about 15 Mbp, only a relatively small fraction of DNA isolated from lytic preparations of whole yeast cell colonies contains the human DNA of interest. Thus, we initially label whole yeast DNA from YAC clones to determine chromosome specificity, cytogenetic map position and chimerism status of YAC clones, before optimizing the DNA probes via isolation of the YAC using PFGE and amplification of the human-specific cDNA by degenerate (mixed base) oligonucleotide-primed PCR (DOP-PCR). The following text describes the unpublished preparation of a chromosome 18-specific locus-specific (LSP) DNA probe from the YAC clone 945B6.

Protocol. Briefly, our procedure to produce a chromosome 18-specific LSP probe was comprised of Step 1:Growing the yeast clone 945B6 on AHC agar (BIO 101) for 2-3 days at 30° C.; picking colonies from the plates and culturing the colonies in 5-35 ml AHC media (BIO 101) at 30° C. for 2-3 days.

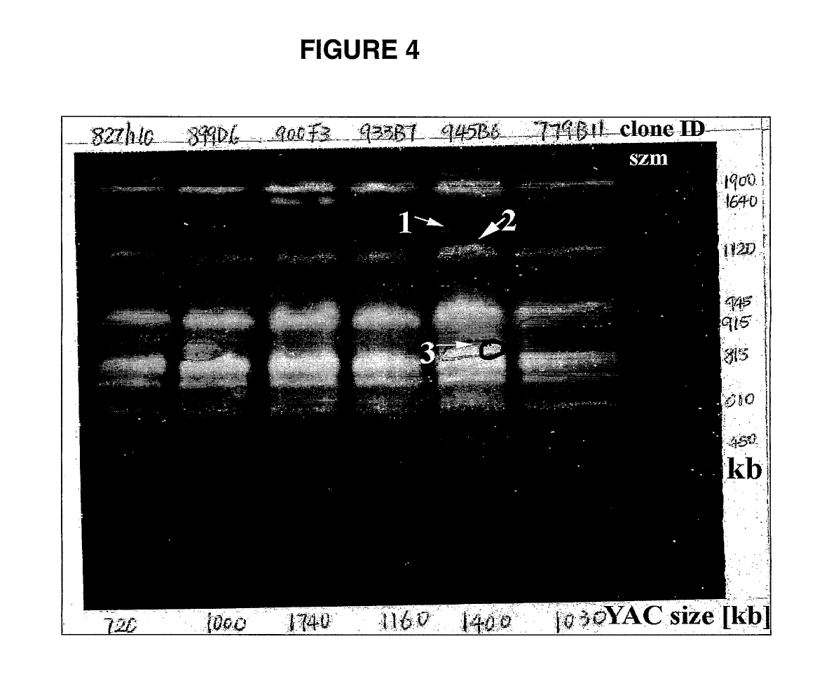

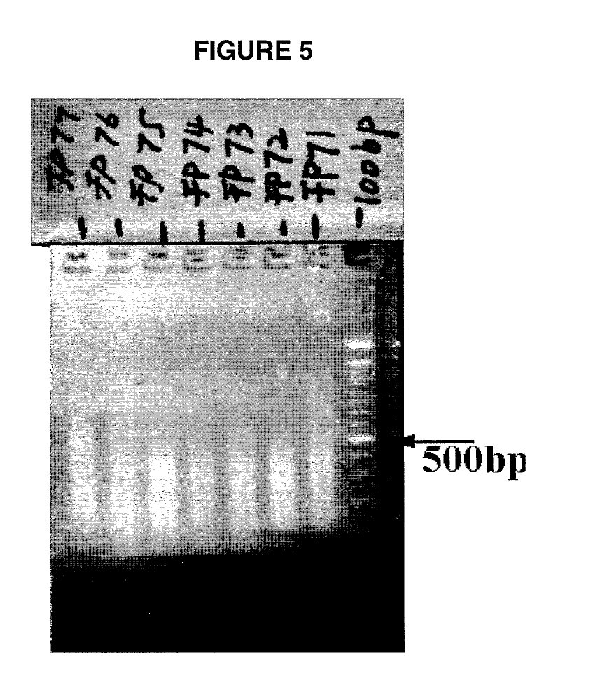

Step 2: Prepare agarose plugs and perform PFGE. This involves (a) pellet the yeast cells from 5 ml AHC medium at 400 rpm for 6 min. Resuspend cells in 0.5 ml EDTA (0.125 M), pH 7.8. Spin down again and remove the supernatant; (b) add 500 μl of SCE (IM sorbitol, 0.1 M Na Citrate, 10 mM EDTA, pH 7.8) to a 70 μl pellet and resuspend. Mix with an equal volume of 1.5% low melting point (LMP) agarose heated to 43° C., quickly pipet up/down, then vortex for 1-2 sec to mix. Pipet into plug molds (BioRad, Inc.) and allow to solidify at 4° C.; (c) remove 4 plugs from molds and incubate plugs in 2 ml SCE with 100 μl of zymolase (10 mg/ml) and shake at 150 rpm at 30° C. for 2.5 hr to overnight; (d) remove SCE and add 2 ml of ES buffer (0.5M EDTA, pH 8.0, 1% Sarcosyl) with 100 μl of proteinase K (20 mg/ml). Shake overnight at 50° C.; (e) remove ES and rinse 5 times with 6 ml of TE50 (10 mM Tris.HCl, 50 ml EDTA, pH 7.8) for 30 min each rinse. Plugs are ready for running PFGE or to be stored at 4° C.; (f) PFGE running conditions: voltage gradient, 6 V/cm; switching interval, 79 sec forward, 94 sec reverse; running time, 38 hr; agarose concentration, 1.0% LMP agarose; running temperature, 14° C.; running buffer, 0.5×TBE; and (f) stain the gel with ethidium bromide (0.5 μg/ml in H₂O) and cut out the YAC band of the target size (1400 kb band marked ‘1’ in FIG. 4) or extra bands, if YAC clone is unstable (FIG. 4, bands ‘2’ and ‘3’).

Step 3: Wash gel slices with water for 30 min, and then equilibrate in 1×β-agarose buffer for 30 min.

Step 4: Melt the gel completely by heating for 10 min at 85° C. Transfer the molten agarose to 43° C. waterbath. Add 1 μl β-agarose for every 25 μl molten agarose and continue incubation for 2 hr. The sample can now be used for the DOP-PCR reactions or stored at 4° C.

Results. FIG. 4 shows a photograph of the PFGE gel containing YAC 945B6 (besides other YAC clones). Four plugs were loaded containing DNA from individual colonies of 945B6. One lane shows a band at 1400 kib as expected for a full length YAC (labeled ‘1’). The other three lanes of 945B6 show smaller inserts. Two additional bands around 1200 kb (labeled ‘2’) and 830 kb (labeled ‘3’) were excised. Only probes prepared from bands 1 and 3 produced signal on chromosome 18p after FISH. DNA from band 2 failed to give FISH signals.

Degenerate Oligonucleotide-Primed PCR (DOP-PCR). We routinely performed two different DNA polymerase chain reactions, an initial amplification reaction using T7 DNA polymerase and primer extension at 37° C. and an automated PCR amplification using Taq polymerase. During the initial 7 cycles of PCR, the DNA is denatured in boiling water for 6 min., before primer JUN1 (5′-CCCAAGCTTGCATGCGAATTCNNNNCAGG-3′, N=A,C,G,T) (SEQ ID NO: 1) is annealed at room temperature. The primer is then extended) for 6 min at 37° C. after addition of one or more units of T7 DNA polymerase (Sequenase II).

The PCR products are then further amplified at higher annealing temperature (50° C.) with primer JUN15 (5′-CCCAAGCTTGCATGCGAATTC-3′) (SEQ ID NO: 2) and Taq polymerase for 35 cycles. We used 10-20 μl of the Sequenase reaction products in a subsequent 200 μl PCR reaction volumes using 4 unit of AMPLITAQ (Roche Molecular). Thermal cycling was performed for 35 cycles on an Perkin Elmer machine (N801-0150) with DNA denaturation at 94° C., primer annealing at 52° C. for 1 min., and primer extension at 72° C. for 2 min. PCR products were analyzed on 2-3% agarose gels (FIG. 2), before they were precipitated in 1.2 volumes isopropanol. Following resuspension of the pellet in 1×TE buffer, the DOP-PCR products were labeled in random priming reactions.

Our strategy for preparation of chromosome 15-specific DNA probes was based in vitro DNA amplification using the polymerase chain reaction (PCR). Oligonucleotide primers were designed based on the known sequence of a highly repeated DNA sequence that maps to the pericentromeric region of human chromosome 15 (Simmons M C, Maxwell J, Haliotis T, Higgins M J, Roder J C, White B N, Holden J J (1984) Amplified KpnL repetitive DNA sequences in homogeneously staining regions of a human melanoma cell line. J. Natl. Cancer Inst. 72(4):801-808). The primers KpnI-F1 (5′-GGGGATCGTTATGGAAAGA-3′) (SEQ ID NO: 3) and KpnI-R1 (5′-TCCATTCCACTCGTTTCCTTT-3′) (SEQ ID NO: 4) were designed to amplify a 159 bp DNA fragment from the larger 1.8 k-b KpnI DNA repeat which maps to the locus D15Z1. D15Z1 was shown to be present at approximately 3,000 copies per haploid genome and organized in long tandem arrays showing restriction site heterogeneity (Higgins M J, Wang H S, Shtromas I, Haliotis T, Roder J C, Holden J J, White B N (1985) Organization of a repetitive human 1.8 kb KpnI sequence localized in the heterochromatin of chromosome 15. Chromosoma. 93(1):77-86).

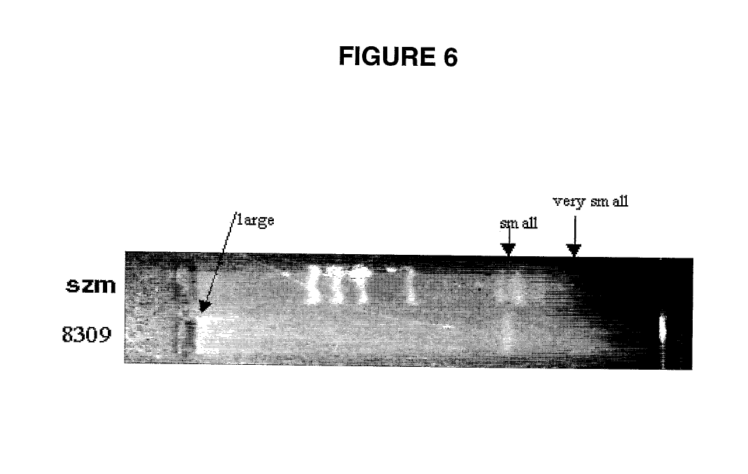

The PCR products (‘8309’) obtained with human genomic DNA as amplification template showed only a very faint band in the 159 bp target size region when analyzed on a 4% agarose gel (FIG. 6, ‘very small’). A stronger band around 300 bp was observed in the same reaction (FIG. 6, ‘small’) as well as some high molecular weight DNA fragments (FIG. 6, ‘large’). We excised the three fractions (very small, small and large) from the gel, melted the agarose in water and performed a second set of PCR reactions using the gel purified DNAs as amplification templates. The DNAs were then labeled by random priming and hybridized to metaphase spreads overnight. The results showed that probe 8309, while giving the brightest signals, also demonstrated heteromorphisms in the cells from some individuals. In these experiments, this probe revealed striking differences in the sizes of hybridization domains representing the hybridization targets on different homologues as well as intensity differences (FIG. 4G). Probes prepared from all three gel purified fractions resulted in somewhat weaker signals. Interestingly, the probes prepared from the 300 bp fragments did not show signs of heteromorphisms and signals from both homologues of chromosome 15 appeared in about the same intensity and size. Thus, by choosing the fraction of probe 8309, we can prepare probes that reveal heteromorphic pattern on chromosome 15 or, although slightly less bright, are not affected by heteromorphisms and thus preferred probes for chromosome enumeration.

### Example 2

### Feasibility of a 8-Probe Set for SIm Technique

We previously constructed a 10-chromosome probe set for detection of DNA targets most frequently associated with aneuploidy and spontaneous abortions (chromosomes 9, 13, 14, 15, 16, 18, 21, 22, X, and Y). Six fluorochromes (Spectrum Green, FITC, Spectrum Orange, Cy3, Cy5, and Cy5.5) were used to detect DNA probes (Fung et al., 2000) (Table 3).

A Spectral Imaging system combines fluorescence spectroscopy and digital imaging for the analysis of FISH signals and was used to score hybridization of the probes to chromosomes in interphase cells. The system was comprised of a fluorescence microscope equipped with an interferometer and a charge-coupled device (CCD) camera plus computer software to perform rapid Fourier spectroscopy. Such systems are commercially available from Applied Spectral Imaging, Carlsbad, Calif.

TABLE 4
Fluorochrome labeling scheme for
chromosome-specific DNA probes*

| Chromo- | Spectrum | | Spectrum | | Biotin | Digoxigenin |
|---|---|---|---|---|---|---|
| some | Green | FITC | Orange | Cy3 | (Cy5) | (Cy5.5) |
| 9 | | + | | | | |
| 13 | | + | | | | + |
| 14 | | + | | + | | |
| 15 | | | + | | | |
| 16 | | | | + | | + |
| 18 | | | | + | + | |
| 21 | | | | | | + |
| 22 | | | | | + | |
| X | + | | + | | | |
| Y | + | | | | | |

*Probes labeled with biotin or digoxigenin were detected with avidin-Cy5 and Cy5.5-conjugated antibodies against biotin or digoxigenin, respectively.

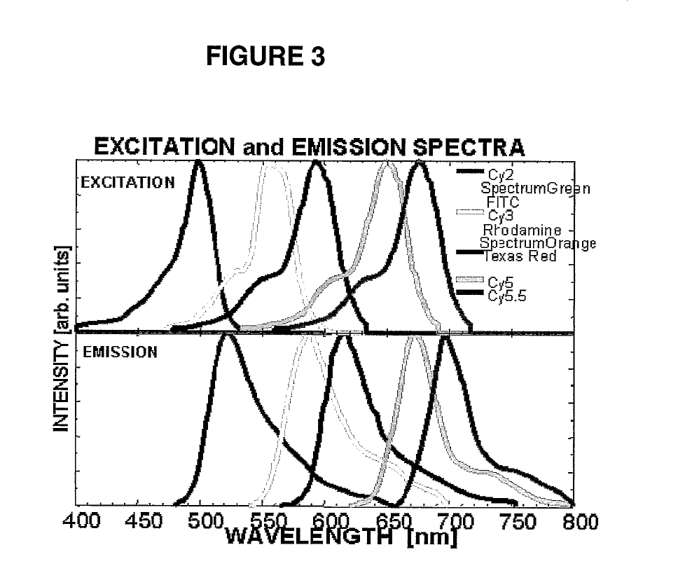

Hybridization of the human chromosomes with 24 chromosome-specific whole chromosome painting (WCP) probes labeled individually with Spectrum Green, Spectrum Orange, Texas Red, Cy5, or Cy5.5 or combinations thereof allowed rapid analysis of metaphase spreads in a single experiment. The multiple band pass filter set (ChromaTechnology, Brattleboro, Vt.) (FIG. 2) used for fluorochrome excitation was custom-designed to provide three broad emission bands centered around 470 nm, 565 nm and 640 nm. Fluorescence was recorded through a multi-bandpass filter with broad transmission peaks in the vicinities of 520 nm (green), 600 nm (red) and 700 nm (infrared, FIG. 2) (Schroeck et al., 1996). This excitation/emission profile matches the five fluorochromes mentioned above (FIG. 3).

Using a Xenon light source, the spectral image was generated by acquiring 80-130 interferometric frames per object. The sample emission spectra (400-850 nm) were measured simultaneously at all points in the microscopic image. The spectral information was displayed by assigning red, green or blue colors to certain ranges of the spectrum. This red, green, blue (RGB)-display renders chromosomes that were labeled with spectrally overlapping fluorochromes or fluorochrome combinations in a similar color. Based on the measurement of the pure spectrum for each chromosome a spectral classification algorithm was applied that allowed the assignment of a pseudo-color to all pixels in the image that have the same fluorescence spectrum. Chromosome identification was then performed by comparison of the measured spectra with pre-recorded reference spectra, and chromosomes were displayed in ‘classification’ colors to facilitate the detection of translocations involving non-homologous chromosomes in SKY analyses of metaphase spreads or as described below the loss of chromosomes or detection of extra chromosomes in SIm analysis of interphase cells (Fung et al., 2000).

As outlined in Table 4, Fung et al. scored chromosomes in interphase cells after hybridization with DNA probes labeled with specific combinations of the 6 fluorochromes. For example, a chromosome 9-specific probe was labeled with FITC, and a chromosome 18-specific probe is labeled with equal parts of Cy3 and Cy5 (Table 4). For each of these probes, we had acquired a reference spectrum using FISH onto metaphase chromosomes. The results demonstrated the unique power of FISH in conjunction with Spectral Imaging for identifying chromosomes in single interphase nuclei (Fung et al., 2000).

### Example 3

### Using Fish Probes to Detect Frequence of Aneuploidy

We investigated the frequencies of abnormalities involving either chromosome 1, 16, 18 or 21 in failed-fertilized human oocytes. While abnormalities involving chromosome 16 showed an age-dependant increase, results for the other chromosomes did not show statistically significant differences between the three age groups <35 yrs, 35-39 yrs, and >39 yrs. Using FISH, we investigated the frequency of aneuploidy and chromatid pre-division for chromosomes 1, 16, 18, and 21 in 273 failed-fertilized oocytes from 95 patients, stratified by age (<35 yrs, 35-39 yrs and >39 yrs; age range 26.1 to 42.2 yrs). Oocytes were prepared as described (Racowsky C, Kaufman M L, Dermer R A, Homa S T, Gunnala S: Chromosomal analysis of meiotic stages of human oocytes matured in vitro: Benefits of protease treatment before fixation. Fertil Steril 1992; 57:1026-1033).

Our probes were labeled with four different fluorochromes, so that they could be identified and scored easily ii the fluorescence microscope (FIG. 7A-D). The DNA probes for chromosomes 1, 18 or 21 were labeled by random priming using a BioPrime kit (Invitrogen, Gaithersburg, Md.) to incorporate digoxigenin-11-dUTP (Roche Molecular Biochemicals, Indianapolis, Ind.), fluorescein-12-dUTP (FITC, Roche Molecular Biochemicals), or Spectrum Orange-dUTP (Vysis, Downers Grove, Ill.). A cloned 1.77-1 kb Eco RI fragment of human satellite III DNA specific for chromosome 1 (pUC1.77)(Fung et al., 2001) was labeled with Spectrum Orange. The probe specific for satellite II DNA of chromosome 16 was prepared from clone pHUR195 (Fung et al., 2001) and labeled with FITC as well as digoxigenin. The single copy DNA probe specific for chromosome 21 was selected from the Genethon/CEPH yeast artificial chromosome (YAC) library (YAC 141G6) and labeled with FITC as previously described (Fung et al., 2001). The DNA probe specific for chromosome 18 (CEP18), labeled with Spectrum Aqua®, was obtained from the manufacturer (Vysis).

We developed a novel chromosome 15-specific DNA repeat probe based on D15Z1 DNA sequence information and in vitro DNA amplification using the polymerase chain reaction (PCR). This probe has the unique feature that it can be tailored to either score copies of chromosomes 15 or detect heteromorphisms involving the locus D15Z1. We also prepared fluorochrome-labeled probes from Pulsed Field Gel Electrophoresis (PFGE) purified yeast artificial chromosome (YAC) clones specific for the human chromosomes 13, 18, 21 or 22. We selected previously cloned DNA repeats for the preparation of blue fluorescent, DEAC-labeled probes for chromosomes 1, 16, X and Y and determined their hybridization strength (FIG. 8B). The three probes specific chromosome for chromosomes 16, X and Y were then labeled with unique fluorochromes.

The results demonstrated an overall high rate of aneuploidy among failed-fertilized eggs in agreement with reports from other laboratories (Martini E, Flaherty S P, Swann N J, Payne D, Matthews C D: Analysis of unfertilized oocytes subjected to intracytoplasmic sperm injection using two rounds of fluorescence in-situ hybridization and probes to five chromosomes. Hum Reprod 1997; 12:2011-2018; Pellestor et al., Maternal aging and chromosomal abnormalities: new data drawn from in vitro unfertilized human oocytes, Hum Genet. 2003 February; 112(2):195-203. Epub 2002 Oct. 29). However, published reports had suggested a disproportionally high rate of aneuploid cells with abnormalities related to the number of copies of chromosome 1 (Martini et al., Hum Reprod 1997; 12:2011-2018; Bahçe et al., Preimplantation genetic diagnosis of aneuploidy: Were we looking at the wrong chromosomes?, J Assist Reprod Genet. 1999; 16:176-181 1986; 85: 1-4). In the study, the rate of cells showing aneusomy involving chromosome 1 was 15.8%, and it was neither age-dependent, nor significantly different from that for chromosomes 16 (22.5%), 18 (16.3%) or 21 (22.7%) (t-test, P=0.241). Table 5 summarizes the chromosome-specific rates of aneuploid cells for each of the three age groups. Only chromosome 16 exhibited a significant age-dependent increase in aneuploidy (12.0%, 30.4% and 50.0% for <35, 35-39 and >39 y; P=0.0009).

TABLE 5
Percentage of cells showing abnormal numbers
of chromosomes 1, 16, 18 and 21

Chromosome

| Age group | 1 | 16 | 18 | 21 |
|---|---|---|---|---|
| <35 yr | 13.3 | 12 | 13.3 | 16 |
| 35-39 yr | 19.6 | 30.4 | 19.6 | 30.4 |
| >39 yr | 16.7 | 50 | 16.7 | 27.8 |

Because chromatid pre-divisions are considered a major mechanism leading to aneuploid oocytes (Pellestor et al., 9003), they were also recorded in our study of failed-fertilized eggs. The rate of chromatid pre-divisions for chromosome 1 was lower than for chromosome 18 (11.9% vs. 25.4%; P=0.01), but not significantly different from that for chromosomes 16 (16.4%) or 21 (19.1%). Moreover, in contrast to previous reports, it was found that the rate of chromatid pre-division involving chromosome 1 did not show any indication of age-dependence. Because the size of the study cohort is in the broad range of the populations reported in the above-cited studies, it is unlikely that the observed discrepancies stem from problems related to cohort size. Other factors such as ethnicity or life style (i.e., diet, caffeine consumption, exercise, smoking, drug use etc.) should be taken into consideration.

Scoring only 4 of the 23 chromosome types found in human oocytes may have underestimated the true incidence of aneuploidy. One approach to determine the number and type of chromosomes in oocytes is based on the delineation of all chromosomes with chromosome-specific whole chromosome painting (WCP) probes. The chromosome-specific WCP probes were prepared by combining probes labeled with one or more of five reporter molecules so that each chromosome-specific WCP probe had a unique identifiable spectral signature. We have applied the SKY technology in the past to demonstrate specific alterations in human and mouse tumors (Fung et al., 2000). In their elegant study, Sandalinas M, Marquez C, Munne S, Spectral karyotyping of fresh, non-inseminated oocytes. Mol Hum Reprod 2002; 8:580-585 33, could demonstrate the application of SI(Y for the analysis of oocytes (FIGS. 7E-G). For chromosome enumeration in oocytes and polar bodies as well as in interphase cell nuclei of other origin, the use of chromosome-specific DNA repeat or single copy probes seems more accurate.

### Example 4

### An Eight Color Set of SIm Probes Used to Detect Aneuploidy Frequency

The scoring of four chromosomes in Example 3 is likely to underestimate the true rate of aneuploid cells. Thus, for a pilot study investigating a more comprehensive analysis of oocytes and their corresponding first polar bodies (1PBs), we developed a novel 8-probe chromosome enumeration scheme using FISH and SIm.

We developed a test system based on 9 color-FISH probes (including the 4′,6-diamidino-2-phenylindole (DAPI) counterstain) and SIm analysis (FIGS. 7H-J). We prepared uniquely labeled DNA probes for simultaneous scoring of chromosomes 13, 15, 16, 18, 21, 22, X, and Y (Table 6). Next, we optimized cell pretreatment and FISH conditions to ensure that each DNA probe reaches a hybridization efficiency of at least 90%, a margin acceptable for most in vitro fertilization (IVF) programs. Probes were tested initially on lymphocytes (FIGS. 7H-J), before use in the analysis of oocytes and their corresponding 1PBs (FIGS. 7K-L).

TABLE 6
The eight color probes set [Set 1].

| target | probe | hybridization | | | | |
|---|---|---|---|---|---|---|
| Xsome | type | strength | clone | label | color | emission |
| 13 | YAC | + | 900G6 | Alexa563 | red | 603 nm |
| 15 | sat II repeat | +++ | uncloned | CY5 | infrared | 667 nm |
| 16 | sat II repeat | ++++ | pHuR195 | CY5.5 | far | 703 nm |
| | | | | | infrared | |
| 18 | repeat | +++ | CEP18 | DEAC | blue | 480 nm |
| 21 | YAC | + | 141G6 | FITC | green | 518 nm |
| 22 | YAC | + | 849E9 | CY3 | orange | 565 nm |
| X | alpha sat | ++ | CEPX | Spectrum Orange | orange | 588 nm |
| Y | sat III repeat | ++++ | pBS124Y-7 | Biotin - Alexa Fluor | green | 539 nm |
| | | | | 635 | | |

We developed the 8 probe set using the probe development protocol in Example 1 and used a SIm-based approach as described in Example 2 to detect bound probes after FISH.

The following is the protocol used for rehybridization testing. Normal male metaphase spreads can be purchased from Vysis, Inc. Vysis, Inc., has implemented very rigorous quality control protocol for their products, and provides high quality metaphase spreads with excellent hybridization efficiency. These slides (ProbeCheck Control slides, cat.# 30-805000, 0% trisomy 8/12 (46XY)) will serve as ‘gold standard’ to measure the efficiency of our 8/24 chromosome scoring protocol. Vysis also sells control slides with an abnormal genotype (Positive Control 10% trisomy 8/12, cat.#30-805002) that we consider using to test our chromosome scoring system. These controls are prepared from cultured normal lymphoblast cell lines or mixtures of a cultured normal lymphoblast cell line and an aneuploid lymphoblast cell line. The cell lines are harvested, fixed in suspension medium, and applied to glass microscope slides in a method optimal for interphase FISH (according to Vysis). These controls demonstrate positive results for trisomy 8 and trisomy 12, and may be used for direct comparison to suspected abnormal cells.

With regard to hybridization, the 8 chromosome-specific probe set (‘Set 1’) is applied to each slide before adding a coverslip. The cells and probe DNA are co-denatured on a hot plate for 10 min at 85° C. After hybridizing overnight at 37° C., the slides are washed in 0.7×SSC (4 min each at 71° C.). The biotin-labeled probes are detected with avidin-Cy3.5 (2.0 mg/ml in PNM), and digoxigenin-labeled probes are detected with (a cross-reacting) Cy5.5-conjugated anti-digoxin antibody (2.0 μg/ml in PNM, Sigma) (Fung et al., 2000). Fluorochrome-labeled probes will not need antibodies or other steps for their detection. The slides are then washed three times in 2×SSC for 10 min each. Finally, the slides are mounted in 8 μl DAPI (Cassel et al., 1997) to counterstain the chromosomes. Routinely, signals are detected and slides are scored on a Nikon E800 fluorescence microscope equipped with the appropriate 4-color fluorescence excitation and emission filters (ChromaTechnology).

Results. In this Example, immature, never inseminated frozen oocytes were matured in vitro and fixed. Oocytes and 1PB's were fixed separately, so they could be analyzed independently. The rate of in vitro maturation of the oocytes was low: less than 50% (42/85) of immature eggs extruded a 1PB. In total, 85 eggs were needed to obtain 29 polar bodies and their respective oocytes. These specimens were analyzed with the 8-probe set. Of those 29 polar bodies, four did not have a corresponding oocyte properly fixed or analyzable. In total there were 25 pairs of 1PBs and oocytes analyzable.

The screen dumps in FIGS. 7K,L show the typical steps during the analysis. Counterclockwise shown from the top left are the pseudo RGB-display of the Spectral Image, automated selection of the region of interest (here a close-up of a chromosome 16-specific signal), the overlay of regions of interest with the inverted DAPI image, and the karyotype table. The small image in the lower left shows a comparison of pre-recorded ‘pure dye’ spectra (black line) with the fluorescence spectrum under the cursor (blue line), which is used to check the accuracy of the automated signal classification.

The concordance between the eggs and 1PBs was found to be 92%, thus proving that the probes, the slide pretreatment and detection protocol are ready for PGD applications. One example, is shown in FIGS. 7K-L, where the 1PB (FIG. 7K) contained two chromosomes 21 (i.e., disomy 21), while the corresponding oocyte carried no chromosome 21 (i.e., nullisomy 21). These results are very reliable, since the single copy probe for chromosome 21 was the only green fluorescent probes in our set.

Based on Table 5, the current FISH test involving chromosomes X, Y, 1, 13, 15, 16, 18, 21 and 22, accounts for 25% cumulative aneuploidy events. (By cumulative events we mean the addition of individual aneuploidy rates, disregarding the occurrence of double aneuploidies). If we assume a 2% rate of aneuploidy for those chromosomes not present in Table 5 (2% is the average of aneuploidy rates of those chromosomes analyzed and close in size to those not analyzed), the total cumulative aneuploidy rate for all 23 chromosome-pairs would be around 56%, compared to 25% for the 8 chromosome pairs detected by FISH.

TABLE 7
Pooled specific chromosome aneuploidy rates:

| | # analysed | #aneuploid |
|---|---|---|
| Chromosome | Embryos | Embryos |
| XY | 1308 | 11 (0.8%) |
| 1 | 550 | 11 (2.0%) |
| 4 | 236 | 6 (2.5%) |
| 6 | 194 | 3 (1.5%) |
| 7 | 235 | 6 (2.6%) |
| 13 | 1350 | 38 (2.8%) |
| 14 | 279 | 3 (1.1%) |
| 15 | 638 | 23 (3.6%) |
| 16 | 1209 | 53 (4.4%) |
| 17 | 218 | 6 (2.8%) |
| 18 | 1607 | 24 (1.5%) |
| 21 | 1548 | 57 (3.7%) |
| 22 | 818 | 51 (6.2%) |
| Total | 1607 | 292 |

∞ Double aneuploidies counted twice, once for each chromosome. Tetrasomies and nulisomies were counted as two trisomies and two monosomies, respectively.

In order to maximize the number of expected chromosome abnormalities, oocyte donors will be 3 5 or older. Based on earlier results, these donors will produce about 8 oocytes each, 4 allocated to each group. Therefore to detect at least a p<0.05 with a power of 80% between a test detecting 25% abnormal oocytes and another detecting 56% we will need about 40 oocytes in each arm, or 10 donors, total.

### Example 5

### Methods for Testing Different Sets of SIm Probes

To determine the efficiency of sequential hybridizations and validation that three sets can be used to analyze a single cell, cells were hybridized with Set 1, analyzed, and rehybridized and analyzed with Set 1 twice more. This was performed on lymphocytes.

Once the probes for Set 1 were designed, each one of them was tested individually in lymphocytes, then mixed in an 8-chromosome cocktail probe and tested in lymphocytes. After that, they were used repeatedly on the same cells to determine the efficiency of rehybridization. It was determined that rehybridization using 3 sets of 8 hybridization probes can be done as described in this Example and Example 4.

Probes for all 24 human chromosomes can be made according to the methods described herein and known in the art. It is preferred that the probes described in Table 2 or Table 3 are used for detection.

First, a reference spectra database for the novel probes will be built. Essentially the same hybridization protocol used to score chromosomes in interphase lymphocytes will be applied, although custom blocking agents for the DNA probe sets may have to prepared. The initial probe tests will use publicly available cells lines from which we will prepare metaphase spreads for probe mapping. We will then test our assay on control slides purchased from Vysis. We will hybridize the first set of probes, acquire Spectral Images from different parts of the slide. To remove the probes, we could use 3 stringent washes in 2×SSC at 73° C. for 10 min each (Walch et al., Sequential multilocus fluorescence in situ hybridization can detect complex patterns of increased gene dosage at the single cell level in tissue sections, Lab Invest. 2001 October; 81(10):1457-9), and then hybridize the next set, but to minimize DNA loss during washes, we have observed that the probes of the first hybridization disappear already during the denaturation required for the second hybridization. Thus the unwashed slide will be co-denatured with the second set of probes and hybridized. This cycle will be repeated once more before we analyze the images. If necessary, we will optimize fixation or hybridization parameters. Among the parameters that can be optimized are hybridization time and temperature, hybridization buffer (incl. several commercial formulations), probe concentration and blocking.

These developments will result in a bio-imaging system including reagents and protocols that delivers signals for at least 90% of expected target chromosomes per set (85% or more overall efficiency for all 24 targets) when using normal cells. Although we target a much larger number of chromosomes, these efficiencies should compare well with studies using 1-5 chromosome-specific probes (See Munné and Weier, 1996).

### Example 5

### Determining Aneuploidy in Reproductive Cells of Patients Having Undergone Chemotherapy

Others have evaluated sperm aneuploidy using multi-colour fluorescence in situ hybridization (FISH), and found approximately 5-fold increases in sperm with disomies, diploidies and complex genotypes involving chromosome X, Y and 8. (Robbins W. A., et al., (1997) Chemotherapy induces transient sex chromosomal and autosomal aneuploidy in human sperm. Nature Genetics 16:74-78.) The aneuploidy effects were transient, however, declining to pretreatment levels within approximately 100 days after the end of the therapy.

Patient samples will be gathered using proper methods and including those described in Robbins et al., Nature Genetics 16:74-78, 1997. Using the present 24 probe set described in Tables 2 or 3 and using the methods in Example 5, the sperm of young men having undergone chemotherapy can be evaluated.

While the present probes, compositions, methods and processes have been described with reference to specific details of certain exemplary embodiments, it is not intended that such details be regarded as limitations upon the scope of the invention. The present examples, methods, procedures, specific sequences and probes are meant to exemplify and illustrate the invention and should in no way be seen as limiting the scope of the invention. Any patents or publications mentioned in this specification and below are indicative of levels of those skilled in the art to which the invention pertains and are hereby incorporated by reference to the same extent as if each was specifically and individually incorporated by reference.

### REFERENCES

Grifo J A, Boyle A, Fischer E, Lavy G, DeCherney A H, Ward D C, Sanyal M K. Abstract Preembryo biopsy and analysis of blastomeres by in situ hybridization. Am J Obstet Gynecol. 1990 December; 163(6 Pt 1):2013-9.
Grifo J A, Boyle A, Tang Y X, Ward D C. Preimplantation genetic diagnosis. In situ hybridization as a tool for analysis. Arch Pathol Lab Med. 1992 April; 116(4):393-7.
Speicher M R, Gwyn Ballard S, Ward D C. Karyotyping human chromosomes by combinatorial multi-fluor FISH. Nat. Genet. 1996 April; 12(4):368-75.
Cassel, M. J., Munné, S., Fung, J., Weier, H.-U. G. (1997) Carrier-specific breakpoint-spanning DNA probes for pre-implantation genetic diagnosis [PGD] in interphase cells. Hum Reprod 12:101-109
Fung, J., Munné, S., Duell, T., Weier, H.-U. G. (1998) Rapid Cloning of Translocation Breakpoints: from Blood to YAC in 50 Days. J Biochem Mol Biol Biophys 1:181-192
Fung, J., Hyun, W., Dandekar, P., Pedersen, R. A., Weier, H.-U. G. (1998) Spectral Imaging in Preconception/Preimplantation Genetic Diagnosis (PGD) of Aneuploidy: Multi-Colour, Multi-Chromosome Screening of Single Cells. J Ass Reprod Genet. 15:322-329
Munné, S., Fung, J., Cassel, M. J., Marquez, C., Weier, H.-U. G. (1998) Preimplantation Genetic Analysis of Translocations: Case-Specific Probes for Interphase Cell Analysis. Human Genetics 102:663-674.
Weier, H.-U. G., Munné S., Fung J. (1999) Patient-specific Probes for Preimplantation Genetic Diagnosis (PGD) of Structural and Numerical Aberrations in Interphase Cells. Journal of Assisted Reproduction and Genetics 16:182-189
Weier, H. U. G., S. Munné, R. A. Lersch, C. Marquez, J. Wu, R. A. Pedersen, J. Fung. (1999) High performance analysis of single interphase cells with custom DNA probes spanning translocation breakpoints. Proc. of SPIE 3604: 227-236
Fung, J., H. U. G. Weier, J. D. Goldberg, R. A. Pedersen (1999) Simultaneous scoring of 10 chromosomes (9, 13, 14, 15, 16, 18, 21, 22, X, Y) in interphase nuclei by using Spectral Imaging. Proc. of SPIE 3604:218-226
Fung J., Munné S., Garcia J., Kim U.-J., Weier H.-U. G. (1999) Reciprocal translocations and infertility: molecular cloning of breakpoints in a case of constitutional transcolation t(11;22)(q23;q11) and preparation of probes for preimplantation genetic diagnosis (PGD). Reproduction, Fertility and Development 11, 17-23.
Weier H.-U. G., Smida J., Zitzelsberger H., Lersch R. A., Hung J., Hsieh H. P., Salassidis K., McNamara G., Pedersen R. A., Fung J. (2000) Cytogenetic Analysis of Interphase Cells using Spectral imaging Technology. Proc. of SPIE 3920:76-85
Lin, S. D., Cooper P., Fung J., Weier, H. U., Rubin E. M. (2000) Genome scan identifies a locus affecting gamma-globulin levels in human beta-cluster YAC transgenic mice. Mamm Genome 11:1024-1029
Lersch R. A., J. Fung, S. Munné, R. A. Pedersen, H.-U. G. Weier (2000) Case-specific, breakpoint-spanning DNA probes for analysis of single interphase cells. Genetic Testing 4:273-278
Fung J, Weier H-U G, Goldberg J D, Pedersen R A (2000) Multilocus genetic analysis of single interphase cells by Spectral Imaging. Hum Genetics 107:615-622
Fung, J., S. Munné, H. U. G. Weier (2001) Detection of Chromosome Translocation Products in Single Interphase Cell Nuclei. Methods in Cell Biology, Vol. 64, Part B, Cytometry, Third Edition (Z. Darzynlciewicz, H. A. Chrissman and J. P. Robinson, Eds.) Academic Press, San Diego, pp. 98-117
Fung J, Weier H-U G, Pedersen R A (2001) Detection of Structural and Numerical Chromosome Abnormalities in Interphase Cells Using Spectral Imaging. J Histochem Cytochem 49:797-798
Weier, H.-U. G., Munné, S., Lersch, R. A., Hsieh, H. B., Smida, J., Chen, X.-N., Korenberg, J. R., Pedersen, R. A., Fung J. (2001) Towards a Full Karyotype Screening of Interphase Cells: FISH and ChipTechnology. Molecular and Cellular Endocrinology 183, Suppl 1:S41-45
Fung J, Weier H-U G, Pedersen R A, Zitzelsberger H F (2002) Spectral Imaging Analysis of Metaphase and Interphase Cells. In: FISH Technology. B. Rautenstrauss and T. Liehr (Eds.), Springer Verlag, Heidelberg, pp. 363-387
Zitzelsberger H F, O'Brien B, Weier HUG (2002) Multicolor FISH techniques for the detection of inter- and intrachromosomal rearrangements. In: FISH Technology. B. Rautenstrauss and T. Liehr (Eds.), Springer Verlag, Heidelberg, pp. 408-424
Weier H-U G, Greulich-Bode K M, Ito Y, Lersch R A, Fung J (2002) Fluorescence in situ hybridization (FISH) in cancer diagnosis and prognostication: from the cause to course of the disease. Expert Review in Molecular Diagnostics 2(2): 109-119.
Liehr, T.; Weise, A.; Heller, A.; Starke, H.; Mrasek, K.; Kuechler, A.; Weier, H.-U. G.; Claussen, U. (2002) Multicolor chromosome banding (MCB) with YAC/BAC-based probes and region-specific microdissection DNA libraries. Cytogenet Genome Res 97:43-50
Weier H-U G, Weier J F, Oter Renom M, Zheng X, Colls P, Nureddin A, Pham C D, Chu L W, Racowsky C, Munné (2005) Fluorescence in situ Hybridization (FISH) and Spectral Imaging (SIm) Analysis of Human Oocytes and First Polar Bodies. J Histochem Cytochem 53: 269-272

TABLE 3

| | | | probe | | probe | | end | end | full |
|---|---|---|---|---|---|---|---|---|---|
| chr. | band | position | name | label | type | clones/DNA | sequ. 1 | sequ. 2 | sequ. ID |
| 13 | q21.31 | 62.520-63.638 | pool3 | red | BAC pool | RP11-527N12 | — | — | AL354810 |
| | | | | | of 8 BACs | RP11-282D7 | — | — | AL355609 |
| | | | | | | RP11-320N6 | — | — | AL359208 |
| | | | | | | RP11-67L17 | — | — | AL354774 |
| | | | | | | RP11-473M10 | — | — | AL445989 |
| | | | | | | RP11-394A14 | — | — | AL445238 |
| | | | | | | RP11-520F9 | — | — | AL355879 |
| | | | | | | RP11-205B18 | — | — | AL354736 |
| 15 | q25.3 | 85.745-86.975M | NTRK3ext | aqua | BAC pool | RP11-116G21 | AQ348695 | AQ348692 | — |
| | | | | | of 7 BACs | RP11-113C11 | AQ344985 | AQ344986 | — |
| | | | | | | RP11-96B23 | AQ313684 | AQ313681 | — |
| | | | | | | RP11-114I9 | AQ344858 | AQ344856 | — |
| | | | | | | RP11-285I14 | — | — | AC011966 |
| | | | | | | RP11-427O16 | — | — | AC023844 |
| | | | | | | RP11-356B18 | — | — | AC009711 |
| | | | | | | RP11-247E14 | — | — | AC087593 |
| | | | | | | RP11-97O12 | — | — | AC013489 |
| 16 | qh, sat II | — | RMC16L006 | infrared 2 | plasmid | RMC16L006 | — | — | X06138 |
| 18 | cen, α-sat | — | D18Z1 | orange | PCR | genomic DNA | — | — | M65181 |
| | | | | | product | | | | |
| 21 | q22 | around 37.770M | D21S167 | green | PCR | YAC 141G6 | — | — | X52289 |
| | | | | | product | | | | |
| 22 | q11.22-q11.23 | 21.545-22.085 | BCR | gold | BAC pool | RP11-357H16 | AZ518881 | AQ553050 | — |
| | | | | | of 3 BACs | RP11-62K15 | AQ199674 | AQ199676 | — |
| | | | | | | RP11-164N13 | AQ380113 | AQ380117 | — |
| X | q21 | 82.447-82.915M | CYCL1 | infrared 1 | BAC pool | RP11-422C9 | AQ553389 | AQ553391 | — |
| | | | | | of 3 BACs | RP11-475A12 | AQ635998 | AQ636000 | — |
| | | | | | | RP11-14A18 | B76225 | B82244 | — |
| Y | q12 | 57.158-57.256M | RP11-242E13 | blue | single BAC | RP11-242E13 | — | — | AC068123 |
| 14 | q11.2 | around 35.279M | D14S75 | red | PCR | YAC 886A3 | — | — | Z16978 |
| | | | | | product | | | | |
| 10 | cen, α-sat | — | 609/51 | aqua | plasmid | 609/51 | — | — | X63622 |
| 19 | q13.2 | around 46.500M | AXL | infrared 2 | BAC pool | CTD-2052L21 | AQ270406 | AQ235108 | — |
| | | | | | of 6 BACs | CTD-2288H11 | B98832 | ? | — |
| | | | | | | CTD-2195C23 | ? | ? | — |
| | | | | | | CTD-2017C04 | ? | ? | — |
| | | | | | | CTD-2195B23 | — | — | AC011510 |
| | | | | | | CTD-2218D21 | AQ032786 | ? | — |
| 17 | cen, α-sat | — | D17Z1 | orange | PCR | RP11-285M22 | — | — | AC131274 |
| | | | | | product | | | | |
| 20 | p11.1-11.2 | 34.950-36.152M | SRC | green | BAC pool | RP11-298O1 | AQ507400 | AQ507403 | — |
| | | | | | of 9 BACs | RP11-465M13 | AQ636482 | — | — |
| | | | | | | RP11-192N1 | AQ412321 | AQ412322 | — |
| | | | | | | RP11-151C5 | AQ376308 | AQ376305 | — |
| | | | | | | RP11-451G10 | AQ583252 | AQ583256 | — |
| | | | | | | RP11-103B19 | AQ313159 | AQ313162 | — |
| | | | | | | RP11-467A7 | AQ637700 | AZ516714 | — |
| | | | | | | RP11-99B19 | AQ318386 | AQ318387 | — |
| | | | | | | RP11-76O8 | AQ266982 | AQ266948 | — |
| 11 | cen, α-sat | — | 238′-5′ | gold | plasmid | 238′-5′ | — | — | M21452 |
| 12 | cen, α-sat | — | 444/7 | infrared 1 | plasmid | YAC 770C7 | — | — | G03348 |
| 9 | cen, sat III | — | PCR1131 | blue | PCR | W21R2 | — | — | X06137 |
| | | | | | product | | | | |
| 3 | q27.3 | 188.590-188.976M | BCL6 | red | BAC pool | RP11-567G11 | — | — | AC104635 |
| | | | | | of 6 BACs | RP11-88P6 | — | — | AC018919 |
| | | | | | | RP11-211G3 | — | — | AC072022 |
| | | | | | | RP11-58M14 | AQ199229 | AQ199231 | — |
| | | | | | | RP11-120O8 | AQ350519 | AQ350515 | — |
| | | | | | | RP11-1E24 | B48349 | AQ312932 | — |
| 5 | q23.1 | 116.279-117.541M | 05BP1-S47 | aqua | BAC pool | RP11-23E11 | B86433 | B86434 | — |
| | | | | | of 7 BACs | RP11-254M1 | AQ479016 | AQ479018 | — |
| | | | | | | RP11-464H3 | AQ586366 | AZ515952 | — |
| | | | | | | RP11-133L2 | AQ350910 | AQ350911 | — |
| | | | | | | RP11-59G17 | AQ194868 | AQ194864 | — |
| | | | | | | RP11-42O22 | AQ116158 | AQ046673 | — |
| | | | | | | RP11-185N19 | — | — | AC021224 |
| 8 | cen, α-sat | — | 8B/9 | infrared 2 | plasmid | 8B/9 | — | — | M64779 |
| 4 | q22.1 | 90.168-91.675 | TIGD2 | orange | BAC pool | RP11-502A23 | — | — | AC079141 |
| | | | | | of 10 BACs | RP11-84C13 | — | — | AC104785 |
| | | | | | | RP11-173C9 | — | — | AC105388 |
| | | | | | | RP11-549C16 | — | — | AC093862 |
| | | | | | | RP11-15F14 | B76416 | B76417 | — |
| | | | | | | RP11-115D19 | — | — | AC097478 |
| | | | | | | RP11-67M1 | — | — | AC093759 |
| | | | | | | RP11-350B19 | — | — | AC105445 |
| | | | | | | RP11-176N15 | — | — | AC108038 |
| | | | | | | RP11-183D16 | — | — | AC093781 |
| 2 | P16.1-15 | 60.525-61-831 | REL | green | BAC pool | RP11-65A9 | AQ237129 | AQ237131 | — |
| | | | | | of 9 BACs | RP11-139C21 | AQ382574 | AQ382578 | — |
| | | | | | | RP11-71D7 | AQ236987 | AQ267891 | — |
| | | | | | | RP11-373L24 | — | — | AC010733 |
| | | | | | | RP11-375M18 | AQ533441 | AQ551245 | — |
| | | | | | | RP11-418N22 | AQ550069 | AQ550072 | — |
| | | | | | | RP11-77P21 | AQ284573 | AQ284575 | — |
| | | | | | | RP11-477N2 | AQ637330 | AQ637326 | — |
| | | | | | | RP11-143D11 | — | — | AC092103 |
| 6 | cen, α-sat | — | PCR199 | gold | PCR | YAC 819A10 | — | — | G04505 |
| | | | | | product | | | | |
| 7 | cen, α-sat | — | 680TA-4 | infrared 1 | plasmid | 680TA-4 | — | — | AJ295152 |
| 1 | p36.33 | around 1.000M | p1-79 | blue | plasmid | p1-79 | — | — | GDB: 157592 |

What is claimed is:

1. A set of 24 chromosome-specific probes to analyze single cells or cell organelles to test for abnormalities in all 24 human chromosomes, wherein the full set comprises three subsets of 8-chromosome-specific probes to analyze single cells or cell organelles.

2. The set of 24 chromosome-specific probes of claim 1, wherein the first subset of 8 chromosome-specific probes is used to test for abnormalities in chromosomes X, Y, 13, 15, 16, 18, 21 and 22.

3. The set of 24 chromosome-specific probes of claim 1, wherein the second subset of 8 chromosome-specific probes is used to test for abnormalities in chromosomes 14, 10, 19, 17, 20, 11, 12 and 9.

4. The set of 24 chromosome-specific probes of claim 1, wherein the third subset of 8 chromosome-specific probes is used to test for abnormalities in chromosomes 1, 2, 3, 4, 5, 6, 7 and 8.

5. The set of 24 chromosome-specific probes of claim 1 used to analyze second polar bodies to test for abnormalities in all 24 human chromosomes.

6. The set of 24 chromosome-specific probes of claim 1 used to analyze blastomeres to test for abnormalities in all 24 human chromosomes.

7. The set of 24 chromosome-specific probes of claim 1 used to analyze spermatocytes to test for abnormalities in all 24 human chromosomes.

8. The set of 24 chromosome-specific probes of claim 1 used to analyze somatic cells to test for abnormalities in all 24 human chromosomes.

9. A method to detect numerical chromosome aberration involving any of the 24 different human chromosome types, comprising the steps of:
(a) providing a single cell or organelle;
(b) treating the single cell or organelle and fixing it to a substrate for analysis to increase accessibility of target DNA and to reduce nonspecific binding;
(c) providing a first subset of 8 probes to detect the target chromosomes;
(d) hybridizing the probes to the target chromosomes in the single cell or organelle;
(e) removing unbound probes; and
(f) detecting the hybridized probes to the target chromosomes, wherein the detection is carried out such that numerical chromosome aberration involving any of the 24 different human chromosome types is detected.

10. The method of claim 9, further comprising the steps of:
(g) removing the hybridized probes from the target chromosomes;
(h) repeating steps (a) through (g) for a second and third subset of probes, wherein each subset of 8 probes detects target chromosomes.

11. The method of claim 9 in which the detecting step is performed using a filter-based fluorescent microscope, optionally equipped with a spectral imaging system.

12. The method of claim 9 in which the detecting step is performed on interphase, non-proliferating or resting cells or organelles.

13. The method of claim 12 in which the detecting step is performed on interphase cells.

14. A set of 24 chromosome-specific probes to analyze tumor cells to test for abnormalities in a human chromosome, wherein the full set comprises three subsets of 8-chromosome-specific probes to analyze tumor cells.
